(19) United States
(12) Patent Application Publication
(10) Pub. No.: US 2026/0002149 A1
(43) Pub. Date: Jan. 1, 2026

(54) LIGATION DEPENDENT DUAL 3'/5' ASSAY FOR SPATIAL AND/OR SINGLE CELL APPLICATIONS

(71) Applicant: 10x Genomics, Inc., Pleasanton, CA (US)

(72) Inventors: Lauren Gutgesell, Fremont, CA (US); Ruijie Zhang, Milpitas, CA (US)

(21) Appl. No.: 19/250,669

(22) Filed: Jun. 26, 2025

Related U.S. Application Data
(60) Provisional application No. 63/665,753, filed on Jun. 28, 2024.

(51) Int. Cl. C12N 15/10

(52) U.S. Cl. CPC C12N 15/1065

(57) ABSTRACT

Provided are methods, systems, and kits for circularization-based dual 3′/5′ assays for sequence analysis of barcoded nucleic acids. The circularization-based dual 3′/5′ assays included single cell sequencing assays and spatial sequencing assays.

### RELATED APPLICATION DATA

This application claims benefit of U.S. Provisional Application No. 63/665,753 filed Jun. 28, 2024, of which is herein incorporated by reference in its entirety.

### FIELD

The present disclosure relates in some aspects to nucleic acid sequencing assays.

### BACKGROUND

Nucleic acid sequencing is a versatile tool that helps scientists advance the understanding of biology and has wide-ranging applications in various fields, such as medical diagnostics, biotechnology, forensic biology, and virology. Reverse transcription-based assays for resolving sequences, such as spatially or at the single cell level, typically obtain sequence information from either 3′ end of the transcript or 5′ end of the transcript. Solutions are needed for obtaining barcode-resolved sequence information from both 3′ end and 5′ end in a single assay.

### SUMMARY

In some aspects, provided herein is a method of preparing a sequencing library including: (a) providing (i) a biological sample comprising a nucleic acid analyte, (ii) a first oligonucleotide comprising at least one barcode sequence, a region that hybridizes to a first portion of the nucleic acid analyte or an extension product thereof, and a first region for self-complementarity, and (iii) a second oligonucleotide comprising a region that hybridizes to a second portion of the nucleic acid analyte or extension product thereof, a second region for self-complementarity, and a second primer binding site; (b) performing extension reactions comprising: an extension reaction using the first oligonucleotide and the nucleic acid analyte or an extension product thereof, and an extension reaction using the second oligonucleotide and the nucleic acid analyte or an extension product thereof, wherein following the extension reactions, an extended molecule is generated comprising: a sequence of the first oligonucleotide comprising the at least one barcode sequence and the first region for self-complementarity, a sequence of the nucleic acid analyte comprising the first portion and the second portion, and a sequence of the second oligonucleotide comprising the second region for self-complementarity; (c) annealing the first region of self-complementarity to the second region of self-complementarity; (d) ligating a 5′ terminus and a 3′ terminus of the extended molecule to generate a circularized barcoded nucleic acid molecule; and (e) performing an amplification reaction to generate amplicons. In some embodiments, the at least one barcode sequence comprises a first barcode sequence and a second barcode sequence, and wherein the first oligonucleotide comprises a primer region positioned between the first barcode sequence and the second barcode sequence.

In some embodiments, the amplification reaction comprises generating amplicons using a forward primer that binds to the primer region and a reverse primer that binds the primer region.

In some embodiments, the method further includes (f) fragmenting the amplicons to generate first fragments comprising a sequence of the first end of the nucleic acid analyte and the first barcode, and second fragments comprising a sequence of the second end of the nucleic acid analyte and the second barcode. In some embodiments, the method further includes appending sequencing primers to both ends of the first fragments and the second fragments.

In some embodiments, following performing the amplification reaction prior to appending the sequencing primers, the method further comprises performing at least purification reaction on the amplicons and/or fragments.

In some embodiments, the following the fragmenting and prior to the appending the sequencing adapters, the method further includes repairing the ends of the first fragments and the second fragments.

In some embodiments, the method further includes generating a sequencing library.

In some embodiments, the first barcode sequence and the second barcode sequence are identical.

In some embodiments, the first oligonucleotide further comprises at least one unique molecular identifier. In some embodiments, the at least one unique molecular identifier comprises two unique molecular identifiers, optionally wherein the two unique molecular identifiers are identical.

In some embodiments, the extension reaction using the first oligonucleotide is performed before the extension reaction using the second oligonucleotide.

In some embodiments, the nucleic acid analyte is an mRNA. In some embodiments, the region that hybridizes to the first portion of the mRNA or extension product thereof comprises a polyT sequence and the first portion of the mRNA comprises a 3′ polyA sequence.

In some embodiments, the region that hybridizes to the second portion of the mRNA or extension product thereof comprises a polyG sequence, and wherein the mRNA or extension product thereof is the extension product and comprises a non-templated terminal polyC.

In some embodiments, the extensions reaction using the second oligonucleotide is performed before the extension reaction using the first oligonucleotide.

In some embodiments, the region that hybridizes to the second portion of the mRNA or extension product thereof comprises a polyT sequence and the second portion of the mRNA comprises a 3′ polyA sequence.

In some embodiments, the region that hybridizes to the first portion of the mRNA or extension product thereof comprises a polyG sequence, and wherein the mRNA or extension product thereof is the extension product and the first portion of the extension product comprises a non-templated terminal polyC.

In some embodiments, the first oligonucleotide is part of an array.

In some embodiments, the first oligonucleotide is attached to a substrate. In some embodiments, the substrate comprises glass, one or more polymers, a hydrogel, a wafer, a plate, or combinations thereof. In some embodiments, the biological sample is a cell or tissue sample attached to a support, and the at least one barcode is a spatial barcode.

In some embodiments, the substrate comprises a bead, a surface of a well, or a slide.

In some embodiments, the biological sample is a single cell, cell bead, or nuclei, and the biological sample is provided in a partition. In some embodiments, at least one barcode is a partition-specific barcode.

In some embodiments, after (c) and prior to (d), the method further comprises contacting the self-complementary barcoded cDNA molecule with a phosphorylated primer and extending from the phosphorylated primer to generate an extension product of the self-complementary barcoded cDNA.

In some embodiments, the sequence of the nucleic acid analyte is at least 100 nucleotides in length.

In some embodiments, following the extension reactions, the extended molecule generated comprises in 3′ to 5′ or 5′ to 3′ order: the sequence of the first oligonucleotide, the nucleic acid analyte sequence in 3′ to 5′ orientation with respect to the nucleic acid analyte, and the sequence of the second oligonucleotide, wherein a 5′ end of the nucleic acid analyte sequence is adjacent to the sequence of the second oligonucleotide.

In some embodiments, the sequence of the nucleic acid analyte is at least 100 nucleotides in length and following the ligating, 5′ end of the nucleic acid analyte sequence is at a proximity of at least 50 nucleotides from a barcode sequence of the at least one barcode sequence in the circularized barcoded nucleic acid molecule.

In some embodiments, following the extension reactions, the extended molecule generated comprises in order: the sequence of the first oligonucleotide, the nucleic acid analyte sequence in 5′ to 3′ orientation with respect to the nucleic acid analyte, and the sequence of the second oligonucleotide, optionally wherein the nucleic acid analyte is an mRNA and the polyA sequence of the mRNA is adjacent to the sequence of the second oligonucleotide.

In some embodiments, the sequence of the mRNA is at least 100 nucleotides in length and following the ligating, the polyA sequence of the mRNA sequence is at a proximity of at least 50 nucleotides from a barcode sequence of the at least one barcode sequence in the circularized barcoded nucleic acid molecule.

In some aspects, provided herein is a method for nucleic acid sequencing in a biological sample, wherein the biological sample is a single cell, the method including: (a) providing a plurality of partitions, wherein a partition of the plurality of partitions comprises: (i) a nucleic acid analyte from the single cell, (ii) a primer molecule, wherein the primer molecule comprises a sequence that hybridizes to the nucleic acid analyte, and (iii) a nucleic acid barcode molecule, wherein the nucleic acid barcode molecule comprises a cell barcode sequence and a template switching oligonucleotide (TSO) comprising a hybridization region at its 3′ end; (b) extending the primer molecule using a reverse transcriptase having terminal transferase activity to generate a cDNA molecule comprising a sequence that is complementary to the hybridization region of the TSO; (c) hybridizing the cDNA molecule to the hybridization region of the TSO; (d) extending the cDNA molecule using the nucleic acid barcode molecule as a template; € circularizing the cDNA molecule to generate a circularized cDNA molecule; and (f) sequencing (i) all or a part of a sequence of the cDNA molecule at its 5′ end, or a complement thereof, (ii) all or a part of a sequence of the cDNA molecule at its 3′ end, or a complement thereof, and (iii) the cell barcode sequence, or a complement thereof.

In some aspects, provided herein is a method for nucleic acid sequencing in a biological sample, wherein the biological sample is a single cell, the method including: (a) providing a plurality of partitions, wherein a partition of the plurality of partitions comprises (i) a nucleic acid analyte from the single cell and (ii) a nucleic acid barcode molecule, wherein the nucleic acid barcode molecule comprises a cell barcode sequence and a hybridization region at its 3′ end; (b) hybridizing the nucleic acid analyte to the hybridization region; (c) extending the nucleic acid barcode molecule using the nucleic acid analyte as a template, thereby generating a cDNA molecule; (d) circularizing the cDNA molecule to generate a circularized cDNA molecule; and €sequencing (i) all or a part of a sequence of the cDNA molecule at its 5′ end, or a complement thereof, (ii) all or a part of a sequence of the cDNA molecule at its 3′ end, or a complement thereof, and (iii) the cell barcode sequence, or a complement thereof.

In some embodiments, all or a part of a sequence of the cDNA molecule at its 5′ end comprises about 50, about 75, about 100, about 150, about 200, about 250, about 300, about 350, about 400, about 450, about 500 or more nucleotides, or all or a part of a sequence of the cDNA molecule at its 3′ end comprises about 50, about 75, about 100, about 150, about 200, about 250, about 300, about 350, about 400, about 450, about 500 or more nucleotides.

In some embodiments, the method further comprising amplifying all or part of the circularized cDNA molecule.

In some embodiments, the nucleic acid barcode molecule further comprises a UMI and/or a primer.

In some embodiments, extending the cDNA molecule utilizes a reverse transcriptase or a polymerase.

In some embodiments, circularizing the cDNA molecule utilizes a ligase. In some embodiments, the ligase is selected from a PBCV-1 DNA ligase, a Chlorella virus DNA ligase, a single stranded DNA ligase, or a T4 DNA ligase.

In some embodiments, the circularized cDNA molecule comprises one or more cell barcodes, wherein the one or more cell barcodes comprises the cell barcode sequence. In some embodiments, the circularized cDNA molecule comprises one or more functional domains, one or more unique molecule identifiers (UMIs), or combinations thereof.

In some embodiments, the one or more functional domains comprise one or more primers.

In some embodiments, the one or more primers amplify the cDNA molecule both at its 5′ end and at its 3′ end, thereby generating a plurality of nucleic acid analyte amplicons.

In some embodiments, plurality of nucleic acid analyte amplicons comprises: a 5′ nucleic acid analyte amplicon comprising sequences of (i) all or part of the sequence of the cDNA molecule at its 5′ end, or a complement thereof, (ii) one or more UMIs, or complements thereof, and (iii) one or more spatial barcodes, or complements thereof; and a 3′ nucleic acid analyte amplicon comprising sequences of (i) all or part of the sequence of the cDNA molecule at its 3′ end, or a complement thereof, (ii) one or more UMIs, or complements thereof, and (iii) one or more spatial barcodes, or complements thereof.

In some embodiments, the plurality of nucleic acid analyte amplicons comprises: a 5′ nucleic acid analyte amplicon comprising sequences of (i) all or part of the sequence of the cDNA molecule at its 5′ end, or a complement thereof, (ii) one UMI, or a complement thereof, and (iii) the spatial barcodes, or a complement thereof; and a 3′ nucleic acid analyte amplicon comprising sequences of (i) all or part of the sequence of the cDNA molecule at its 3′ end, or a complement thereof, (ii) one UMI, or a complement thereof, and (iii) the spatial barcodes, or a complement thereof.

In some embodiments, the hybridization region of the TSO comprises a poly(G) sequence and wherein the nucleic acid barcode molecule comprises a poly(C) sequence.

In some embodiments, the nucleic acid barcode molecule is coupled to a particle. In some embodiments, the particle is a bead. In some embodiments, one or more cells are separated into the plurality of partitions. In some embodiments, the partition is a droplet, microwell, or well.

In some embodiments, the method further comprises permeabilizing the biological sample using a reagent medium. In some embodiments, the reagent medium comprises a protease. In some embodiments, the protease is selected from trypsin, pepsin, elastase, or proteinase K. In some embodiments, the protease is pepsin or proteinase K. In some embodiments, the reagent medium further comprises a detergent. In some embodiments, the detergent is selected from sodium dodecyl sulfate (SDS), sarkosyl, or saponin. In some embodiments, the reagent medium further comprises polyethylene glycol (PEG).

In some embodiments, the nucleic acid analyte comprises RNA. In some embodiments, the RNA is mRNA. In some embodiments, the nucleic acid analyte comprises DNA. In some embodiments, the DNA is genomic DNA.

In some aspects, provided herein is a method of determining location and abundance of a nucleic acid analyte in a biological sample, the method including: (a) contacting the biological sample with a substrate; (b) hybridizing the nucleic acid analyte to a capture domain of a capture probe on an array, thereby generating a captured nucleic acid analyte, wherein the capture probe further comprises a spatial barcode; (c) extending the capture probe using the captured nucleic acid analyte as a template to produce an extended capture probe; (d) synthesizing a second strand using the extended capture probe as a template; (e) circularizing the second strand to generate a circularized second strand; and (f) determining (i) all or a part of a sequence of the captured nucleic acid analyte at its 5′ end, or a complement thereof, (ii) all or a part of a sequence of the captured nucleic acid analyte at its 3′ end, or a complement thereof, and (iii) the spatial barcode, or a complement thereof, and using the determined sequences of (i), (ii), and (iii) to determine the location and abundance of the nucleic acid analyte in the biological sample.

In some embodiments, all or a part of a sequence of the captured nucleic acid analyte at its 5′ end comprises about 50, about 75, about 100, about 150, about 200, about 250, about 300, about 350, about 400, about 450, about 500 or more nucleotides, and/or all or a part of a sequence of the captured nucleic acid analyte at its 3′ end comprises about 50, about 75, about 100, about 150, about 200, about 250, about 300, about 350, about 400, about 450, about 500 or more nucleotides.

In some embodiments, the method further comprises amplifying all or part of the circularized second strand.

In some embodiments, wherein determining (i) all or a part of a sequence of the captured nucleic acid analyte at its 5′ end, or a complement thereof, (ii) all or a part of a sequence of the captured nucleic acid analyte at its 3′ end, or a complement thereof, and (iii) the spatial barcode, or a complement thereof, comprises sequencing.

In some embodiments, the array is on the substrate. In some embodiments, the substrate comprises glass, one or more polymers, a hydrogel, a wafer, a plate, or combinations thereof.

In some embodiments, the array is on a second substrate. In some embodiments, the second substrate comprises glass, one or more polymers, a hydrogel, a wafer, a plate, or combinations thereof. In some embodiments, the method further comprises aligning the substrate with the second substrate comprising the array, such that at least a portion of the biological sample is aligned with at least a portion of the array. In some embodiments, the aligning comprises: mounting the first substrate on a first member of a support device, the first member configured to retain the first substrate; mounting the second substrate on a second member of the support device; applying a reagent medium to the first substrate and/or the second substrate; and operating an alignment mechanism of the support device to move the first member and/or the second member such that at least a portion of the biological sample is aligned with at least a portion of the array, and such that the portion of the biological sample and the portion of the array contact the reagent medium. In some embodiments, the alignment mechanism is coupled to the first member, the second member, or both the first member and the second member. In some embodiments, the alignment mechanism comprises a linear actuator, optionally wherein: the linear actuator is configured to move the second member along an axis orthogonal to the first member and/or the second member, and/or the linear actuator is configured to move the first member along an axis orthogonal to a plane of the first member and/or the second member, and/or the linear actuator is configured to move the first member, the second member, or both the first member and the second member at a velocity of at least 0.1 mm/sec, and/or the linear actuator is configured to move the first member, the second member, or both the first member and the second member with an amount of force of at least 0.1 lbs.

In some embodiments, at least one of the first substrate and the second substrate further comprise a spacer disposed on the first substrate or the second substrate, wherein when at least the portion of the biological sample is aligned with at least a portion of the array such that the portion of the biological sample and the portion of the array contact the reagent medium, the spacer is disposed between the first substrate and the second substrate and is configured to maintain the reagent medium within a chamber formed by the first substrate, the second substrate, and the spacer, and to maintain a separation distance between the first substrate and the second substrate, wherein the spacer is positioned to surround an area on the first substrate on which the biological sample is disposed and/or the array disposed on the second substrate, wherein the area of the first substrate, the spacer, and the second substrate at least partially encloses a volume comprising the biological sample.

In some embodiments, extending the capture probe utilizes a reverse transcriptase or a polymerase. In some embodiments, synthesizing the second strand utilizes a polymerase.

In some embodiments, the method further comprises separating the second strand from the extended capture probe. In some embodiments, separating the second strand from the extended capture probe comprises adding potassium hydroxide to the substrate.

In some embodiments, circularizing the second strand utilizes a ligase. In some embodiments, the ligase is selected from a PBCV-1 DNA ligase, a Chlorella virus DNA ligase, a single stranded DNA ligase, or a T4 DNA ligase.

In some embodiments, the circularized second strand comprises one or more spatial barcodes, wherein the one or more spatial barcodes comprises the spatial barcode. In some embodiments, the circularized second strand comprises one or more functional domains, one or more unique molecule identifiers (UMIs), or combinations thereof. In some embodiments, the one or more functional domains comprise one or more primers.

In some embodiments, the one or more primers amplify the captured nucleic acid analyte both at its 5′ end and at its 3′ end, thereby generating a plurality of nucleic acid analyte amplicons. In some embodiments, the plurality of nucleic acid analyte amplicons comprises: a 5′ nucleic acid analyte amplicon comprising sequences of (i) all or part of the sequence of the captured nucleic acid analyte at its 5′ end, or a complement thereof, (ii) one or more UMIs, or complements thereof, and (iii) one or more spatial barcodes, or complements thereof; and a 3′ nucleic acid analyte amplicon comprising sequences of (i) all or part of the sequence of the captured nucleic acid analyte at its 3′ end, or a complement thereof, (ii) one or more UMIs, or complements thereof, and (iii) one or more spatial barcodes, or complements thereof.

In some embodiments, the plurality of nucleic acid analyte amplicons comprises: a 5′ nucleic acid analyte amplicon comprising sequences of (i) all or part of the sequence of the captured nucleic acid analyte at its 5′ end, or a complement thereof, (ii) one UMI, or a complement thereof, and (iii) the spatial barcodes, or a complement thereof; and a 3′ nucleic acid analyte amplicon comprising sequences of (i) all or part of the sequence of the captured nucleic acid analyte at its 3′ end, or a complement thereof, (ii) one UMI, or a complement thereof, and (iii) the spatial barcodes, or a complement thereof.

In some embodiments, the capture probe further comprises one or more functional domains, a UMI, a cleavage domain, or combinations thereof. In some embodiments, wherein the one or more functional domains comprises a primer binding site. In some embodiments, wherein the capture domain comprises a homopolymeric sequence. In some embodiments, wherein the capture domain comprises a poly(T) sequence.

In some embodiments, the nucleic acid analyte comprises RNA. In some embodiments, the RNA is mRNA. In some embodiments, the nucleic acid analyte comprises DNA. In some embodiments, wherein the DNA is genomic DNA.

In some embodiments, the biological sample is a tissue sample. In some embodiments, the tissue sample is a tissue section. In some embodiments, the biological sample is a fresh tissue sample and/or a frozen tissue sample. In some embodiments, the biological sample is a fixed tissue sample. In some embodiments, the fixed tissue sample is a formalin fixed paraffin embedded (FFPE) tissue sample. In some embodiments, the FFPE tissue sample is deparaffinized and decrosslinked. In some embodiments, the biological sample is a suspension of cells or a culture of cells. In some embodiments, the biological sample is stained. In some embodiments, the biological sample is stained using immunofluorescence, immunohistochemistry, hematoxylin, and/or eosin.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein entirely incorporated by reference for all purposes to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual analyte carriers.

FIG. 2 shows an example of a microfluidic channel structure for the controlled partitioning of beads into discrete droplets.

FIG. 3 shows an exemplary microfluidic channel structure for delivering barcode carrying beads to droplets.

FIG. 4 illustrates an example of a barcode carrying bead.

FIG. 5 illustrates another example of a barcode carrying bead.

FIG. 6 schematically illustrates an example microwell array.

FIG. 7 schematically illustrates an example workflow for processing nucleic acid molecules.

FIG. 8 schematically illustrates example labelling agents with nucleic acid molecules attached thereto.

FIG. 9A schematically shows an example of labelling agents. FIG. 9B schematically shows another example workflow for processing nucleic acid molecules. FIG. 9C schematically shows another example workflow for processing nucleic acid molecules.

FIG. 10 schematically shows another example of a barcode-carrying bead.

FIG. 11A shows an exemplary sandwiching process where a first substrate (e.g., a slide), including a biological sample, and a second substrate (e.g., array slide) are brought into proximity with one another.

FIG. 11B shows a fully formed sandwich configuration creating a chamber formed from the one or more spacers, the first substrate, and the second substrate.

FIG. 12A shows a perspective view of an exemplary sample handling apparatus in a closed position.

FIG. 12B shows a perspective view of an exemplary sample handling apparatus in an open position.

FIG. 13A shows the first substrate angled over (superior to) the second substrate.

FIG. 13B shows that as the first substrate lowers, and/or as the second substrate rises, the dropped side of the first substrate may contact a drop of reagent medium.

FIG. 13C shows a full closure of the sandwich between the first substrate and the second substrate with one or more spacers contacting both the first substrate and the second substrate.

FIG. 14A shows a side view of the angled closure workflow.

FIG. 14B shows a top view of the angled closure workflow.

FIG. 15 is a schematic diagram showing an example of a barcoded capture probe, as described herein.

FIG. 16 shows a schematic illustrating a cleavable capture probe.

FIG. 17 shows exemplary capture domains on capture probes.

FIG. 18 shows an exemplary arrangement of barcoded features within an array.

FIG. 19A provides a schematic of an example preparation of an extension product for single cell circularization-based dual 3′/5′ sequencing of a nucleic acid analyte.

FIG. 19B provides a schematic of an example circularization of an extension product.

FIG. 20 provides a schematic of an example preparation of an extension product for a spatial circularization-based dual 3′/5′ sequencing of a nucleic acid analyte.

FIG. 21 provides a schematic example preparation of an extension product for a circularization-based dual 3′/5 sequencing of a nucleic acid analyte in an alternative orientation.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise. “A and/or B” is used herein to include all of the following alternatives: “A”, “B”, “A or B”, and “A and B”.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value or rounded to the nearest significant figure, in all cases inclusive of the provided value.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include polynucleotide barcodes, random nucleic acid and/or amino acid sequences, and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses). The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.; as well as invertebrates, such as annelids, echinoderms, cnidarians, gastropods, crustaceans, cephalopods, mollusks, Porifera sponges, arachnids, and insects.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. The nucleic acid barcode molecule may be coupled to or attached to the nucleic acid molecule comprising the nucleic acid sequence. For example, a nucleic acid barcode molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle” may be used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a nucleus of a cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell but may not include other constituents of the cell. An example of such constituents is a nucleus or an organelle. A cell may be a live cell. The live cell may be capable of being cultured, for example, being cultured when enclosed in a gel or polymer matrix or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition can be a physical container, compartment, or vessel, such as a droplet, a flowcell, a reaction chamber, a reaction compartment, a tube, a well, or a microwell. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

### I. Overview

Provided herein are methods of preparing a sequencing library such that barcode information (e.g., a single cell barcode or a spatial barcode) attached to one end of a nucleic acid analyte (e.g., a 5′ end or a 3′ end) is utilized for barcode-associated sequencing of both ends of the nucleic acid analyte. These methods may be useful, for example, for generating sequencing reads, which are shorter than the full length sequence of the nucleic acid analyte, but include barcode information at both ends of the nucleic acid analyte.

### Methods for Dual 3′/5′ Analyte and Barcode Sequencing

In some aspects, the present disclosure provides a method of preparing a sequencing library from a biological sample including a nucleic acid analyte (e.g. an nucleic acid analyte). In some embodiments, the method utilized a first oligonucleotide comprising at least one barcode sequence, a region that hybridizes to a first end of the nucleic acid analyte or an extension product thereof, and a first region for self-complementarity, and a second oligonucleotide comprising a region that hybridizes to a second end of the nucleic acid analyte (e.g., mRNA) or an extension product thereof, a second region for self-complementarity, and a second primer binding site. In some embodiments, the method includes performing extension reactions including: a first extension reaction using the first oligonucleotide and the nucleic acid analyte or an extension product thereof, and an extension reaction using the second oligonucleotide and the nucleic acid analyte or an extension product thereof, wherein following the extension reactions, an extended molecule is generated comprising: a sequence of the first oligonucleotide comprising the at least one barcode sequence and the first region for self-complementarity, a sequence of the nucleic acid analyte A, and a sequence of the second oligonucleotide comprising the second region for self-complementarity. In particular embodiments, the extended molecule is a single-stranded nucleic acid molecule. In particular embodiments, the extended molecule is a single-stranded DNA molecule.

In some aspects, following the extension reactions, the method includes annealing the first region of self-complementarity to the second region of self-complementarity. An annealing step may include subjecting the reaction to a temperature under which the self-complementary ends are capable of annealing.

In some aspects, the method includes ligating a 5′ terminus and a 3′ terminus of the extended molecule to generate a circularized barcoded nucleic acid molecule. A 5′ phosphate at the 5′ terminus and a 3′ hydroxy at a 3′ terminus may be ligated using methods known in the art. In some aspects, the ligation is a Y-ligation, wherein a free 5′ end and free 3′ end are ligated proximal to a double-stranded region (e.g., the self-complementary region). In some aspects, the double-stranded region is at the 5′ terminus and the 3′ terminus of the extended product. In some aspects, one, two, three, four, or five nucleotides are single stranded between the double-stranded region and the 5′ terminus. In some aspects, one, two, three, four, or five nucleotides are single stranded between the double-stranded region and the 3′ terminus. Including single-stranded nucleotides beyond the double-stranded region will result in a “bubble” or hairpin region following ligation of the 5′ terminus to the 3′ terminus. Examples of ligases include a PBCV-1 DNA ligase, a Chlorella virus DNA ligase, a single stranded DNA ligase, and a T4 DNA ligase. In some aspects, the ligase is T4 ligase, which is a ligase capable of Y-ligation. In some aspects, the ligation includes adding a short (e.g., less than 12 nucleotides long) double-stranded oligo for ligating to the free 5′ terminus and 3′ terminus of the extended product. In some embodiments, the ligation is blunt ligation. In some embodiments, the extended product includes a single A overhang at the 3′ terminus, and the double-stranded oligo for ligating includes a T overhand at a 5′ terminus.

In some aspects, the method includes performing an amplification reaction on the circularized barcoded nucleic acid molecule using at least one primer set to generate amplicons.

In aspects wherein the at least one barcode includes a first barcode sequence and a second barcode sequence, a first primer binding site and a second primer binding site may be positioned between the first barcode sequence and the second barcode sequence. In some embodiments, the amplification reaction includes generating amplicons using a forward primer that binds to the first primer binding site and a reverse primer that binds the second primer binding site.

In some embodiments, the method further comprises (f) fragmenting the amplicons to generate first fragments comprising a sequence of the first portion of the nucleic acid analyte and the first barcode, and second fragments comprising a sequence of the second portion of the nucleic acid analyte and the second barcode. Fragmentation methods include enzymatic fragmentation and mechanical methods such as sonication. In some embodiments, the fragmentation pattern is random (not sequence specific). Enzymatic and mechanical methods may be used for random fragmentation.

In some embodiments, the method further includes appending sequencing primers or partial sequencing primers (e.g., R1 or partial R1, and R2 or partial R2) to both ends of the first fragments and the second fragments. Appending sequencing primers may be performed by ligation of sequencing adapters, and/or by PCR addition. In some embodiments, the sequencing primers are added by ligation. In some embodiments, the sequencing primers are added by PCR.

In some embodiments, following performing the amplification reaction and prior to appending the sequencing adapters, the method further includes performing at least one purification reaction on the amplicons and/or fragments. Purification methods include ethanol precipitation, phenol: chloroform purification, size exclusion, and magnetic affinity purification. In some embodiment, the purification include a size exclusion purification.

In some embodiments, following the fragmenting and prior to the appending the sequencing adapters, the method further includes repairing the ends of the first fragments and the second fragments. End repair and A-tailing methods are known in the art and reagents for these steps are commercially available.

In some embodiments, the extension reaction using the first oligonucleotide is performed before the extension reaction using the second oligonucleotide. In some embodiments, nucleic acid analyte is mRNA, and the region that hybridizes to the first end of the mRNA or extension product thereof comprises a polyT sequence and the first end of the mRNA comprises a polyA sequence. In some embodiments, the region that hybridizes to the second end of the mRNA or extension product thereof comprises a polyG sequence, and wherein the mRNA or extension product thereof is the extension product and comprises a non-templated terminal polyC. In some embodiments, the extension product is a reverse transcription product. Reverse transcriptase can add untemplated C nucleotides at the end of a reverse transcription product. In some embodiments, the extensions reaction using the second oligonucleotide is performed before the extension reaction using the first oligonucleotide. In some embodiments, the region that hybridizes to the second portion of the mRNA or extension product thereof comprises a polyT sequence and the second end of the mRNA comprises a 3′ polyA sequence. In some embodiments, the region that hybridizes to the first end of the mRNA or extension product thereof comprises a polyG sequence, and wherein the mRNA or extension product thereof is the extension product and the first end of the extension product comprises a non-templated terminal polyC.

In some embodiments, the one of the first oligonucleotide or the second oligonucleotide is provided attached to a substrate. Examples of substrates include glass, ceramics, one or more polymers, a hydrogel, a wafer, a plate, or combinations thereof. In some embodiments, the substrate is a bead, a surface of a well, or a slide.

In some embodiments, the biological sample is a single cell, cell bead, or nuclei, and the biological sample is provided in a partition, and optionally the at least one barcode is a partition-specific barcode.

In some embodiments, the biological sample is a cell or tissue sample attached to a support, and the at least one barcode is a spatial barcode.

In some embodiments of the method, the method includes contacting the extended product with a phosphorylated primer and extending from the phosphorylated primer to generate a second strand of the extension product.

The nucleic acid analyte may be at least at least 50 nucleotides in length, at least 100 nucleotides in length, at least 150 nucleotides in length, at least 200 nucleotides in length, at least 250 nucleotides in length, or at least 300 nucleotides in length. In some embodiments, the sequence of the nucleic acid analyte is at least 100 nucleotides in length. In some embodiments of the method, the method allows for generating a sequence reads including a first portion or first end (e.g., 3′ end) of the nucleic acid analyte, a barcode (e.g., a single cell barcode or a spatial barcode), and a second portion or second end (e.g., 5′ end) of the nucleic acid analyte and the same barcode. The sequence reads may be a first sequence read with the first portion or end (e.g., 3′ end) of the nucleic acid analyte and the barcode, and a second sequence read with the second portion or end (e.g., 5′ end) of the nucleic acid analyte and the same barcode. For spatial and single cell analysis workflows, a typical read length for sequencing the nucleic acid analyte is fewer than 100 nucleotides. Thus for a nucleic acid analyte at least 100 nucleotides in length, linking a first portion (e.g., 3′ end) with a spatial or single cell barcode and linking a second portion (e.g., 5′ end) with a same spatial or single cell barcode unlocks more sequence information than traditional methods.

In some embodiments, following the extension reactions, the extended molecule generated comprises in 3′ to 5′ or 5′ to 3′ order: the sequence of the first oligonucleotide, the nucleic acid analyte sequence in 3′ to 5′ orientation with respect to the nucleic acid analyte, and the sequence of the second oligonucleotide, wherein a 5′ end of the nucleic acid analyte sequence is adjacent to the sequence of the second oligonucleotide. In some embodiments, the sequence of the nucleic acid analyte is at least 100 nucleotides in length and following the ligating, 5′ end of the mRNA sequence is at a proximity of at least 50 nucleotides from a barcode sequence of the at least one barcode sequence in the circularized barcoded nucleic acid molecule.

In some embodiments, following the extension reactions, the extended molecule generated comprises in order: the sequence of the first oligonucleotide, the nucleic acid analyte sequence in 5′ to 3′ orientation with respect to the nucleic acid analyte, and the sequence of the second oligonucleotide, wherein 3′ sequence (e.g., polyA of an mRNA analyte) of the nucleic acid analyte is adjacent to the sequence of the second oligonucleotide.

The method of claim 15, wherein the sequence of the nucleic acid analyte is at least 100 nucleotides in length and following the ligating, 3′ sequence (e.g., polyA of an mRNA analyte) of the nucleic acid analyte sequence is at a proximity of at least 50 nucleotides from a barcode sequence of the at least one barcode sequence in the circularized barcoded nucleic acid molecule.

In some aspects, methods provided herein utilize an oligonucleotide including at least one barcode. In some embodiments, the at least one barcode includes a first barcode and a second barcode. In some embodiments, the first barcode and the second barcode are identical.

In some embodiments, the oligonucleotide (e.g., nucleic acid barcode molecule) comprises at least one unique molecular identifier. In some embodiments, the first oligonucleotide includes a unique molecular identifier. In some embodiments, the first oligonucleotide includes two copies of a same unique molecular identifier (e.g., separated by a primer binding region).

In some aspects, methods provided herein include use of an oligonucleotide molecule including a barcode sequence (e.g., a nucleic acid barcode molecule). In some embodiments, the oligonucleotide includes one or more barcode sequences. A plurality of oligonucleotide molecules including a barcode may be on a support, such as glass, a hydrogel surface, or a bead. In some embodiments, the oligonucleotide including the barcode is coupled to a bead. The one or more barcode sequences may include sequences that are the same for all or a portion of the nucleic acid molecules coupled to a given bead and/or sequences that are different across all (or a portion of the) nucleic acid molecules coupled to the given bead. The nucleic acid molecule may be incorporated into the bead.

Nucleic acid barcode molecules can comprise one or more functional sequences for coupling to an analyte or analyte tag such as a reporter oligonucleotide. Such functional sequences can include, e.g., a template switch oligonucleotide (TSO) sequence, a primer sequence (e.g., a poly T sequence, or a nucleic acid primer sequence complementary to a target nucleic acid sequence and/or for amplifying a target nucleic acid sequence, a random primer, and a primer sequence for messenger RNA).

In some cases, the nucleic acid barcode molecule can comprise one or more functional sequences, for example, for attachment to a sequencing flow cell, such as, for example, a P5 sequence (or a portion thereof) for Illumina® sequencing. In some cases, the nucleic acid barcode molecule or derivative thereof (e.g., oligonucleotide or polynucleotide generated from the nucleic acid molecule) can comprise another functional sequence, such as, for example, a P7 sequence (or a portion thereof) for attachment to a sequencing flow cell for Illumina sequencing. In some cases, the nucleic acid molecule can comprise an R1 primer sequence for Illumina sequencing. In some cases, the nucleic acid molecule can comprise an R2 primer sequence for Illumina sequencing. In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof.

Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

In some aspects, provided herein are methods that include preparing nucleic acid analytes of a sample for sequencing. A sample may derive from any useful source including any subject, such as a human subject. A sample may comprise material (e.g., one or more biological particles) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, may be obtained for analysis as described herein. For example, a first sample may be obtained from a subject at a first time and a second sample may be obtained from the subject at a second time later than the first time. The first time may be before a subject undergoes a treatment regimen or procedure (e.g., to address a disease or condition), and the second time may be during or after the subject undergoes the treatment regimen or procedure. In another example, a first sample may be obtained from a first bodily location or system of a subject (e.g., using a first collection technique) and a second sample may be obtained from a second bodily location or system of the subject (e.g., using a second collection technique), which second bodily location or system may be different than the first bodily location or system. In another example, multiple samples may be obtained from a subject at a same time from the same or different bodily locations. Different samples, such as different subjects collected from different bodily locations of a same subject, at different times, from multiple different subjects, and/or using different collection techniques, may undergo the same or different processing (e.g., as described herein). For example, a first sample may undergo a first processing protocol and a second sample may undergo a second processing protocol. In another example, a portion of a sample may undergo a first processing protocol and a second portion of the sample may undergo a second processing protocol.

A sample may be a biological sample, such as a cell sample (e.g., as described herein). A sample may include one or more biological particles, such as one or more cells and/or cellular constituents, such as one or more cell nuclei. A sample may be a tissue sample. For example, a sample may comprise a plurality of biological particles, such as a plurality of cells and/or cellular constituents. Biological particles (e.g., cells or cellular constituents, such as cell nuclei) of a sample may be of a single type or a plurality of different types. For example, cells of a sample may include one or more different types or blood cells.

Cells and cellular constituents of a sample may be of any type. For example, a cell or cellular constituent may be a vertebral, mammalian, fungal, plant, bacterial, or other cell type. In some cases, the cell is a mammalian cell, such as a human cell. The cell may be, for example, a stem cell, liver cell, nerve cell, bone cell, blood cell, reproductive cell, skin cell, skeletal muscle cell, cardiac muscle cell, smooth muscle cell, hair cell, hormone-secreting cell, or glandular cell. The cell may be, for example, an erythrocyte (e.g., red blood cell), a megakaryocyte (e.g., platelet precursor), a monocyte (e.g., white blood cell), a leukocyte, a B cell, a T cell (such as a helper, suppressor, cytotoxic, or natural killer T cell), an osteoclast, a dendritic cell, a connective tissue macrophage, an epidermal Langerhans cell, a microglial cell, a granulocyte, a hybridoma cell, a mast cell, a natural killer cell, a reticulocyte, a hematopoietic stem cell, a myoepithelial cell, a myeloid-derived suppressor cell, a platelet, a thymocyte, a satellite cell, an epithelial cell, an endothelial cell, an epididymal cell, a kidney cell, a liver cell, an adipocyte, a lipocyte, or a neuron cell. In some cases, the cell may be associated with a cancer, tumor, or neoplasm. In some cases, the cell may be associated with a fetus. In some cases, the cell may be a Jurkat cell.

A biological sample may include a plurality of cells having different dimensions and features. In some cases, processing of the biological sample, such as cell separation and sorting (e.g., as described herein), may affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process may comprise the use of microfluidics (e.g., to separate biological particles of different sizes, types, charges, or other features).

In an example, a sample comprising one or more cells may be processed to separate the one or more cells from other materials in the sample (e.g., using centrifugation and/or another process). In some cases, cells and/or cellular constituents of a sample may be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process may comprise a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells). Separation of one or more different types of cells may comprise, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method.

For example, a flow cytometry method may be used to detect cells and/or cellular constituents based on a parameter such as a size, morphology, or protein expression. Flow cytometry-based cell sorting may comprise injecting a sample into a sheath fluid that conveys the cells and/or cellular constituents of the sample into a measurement region one at a time. In the measurement region, a light source such as a laser may interrogate the cells and/or cellular constituents and scattered light and/or fluorescence may be detected and converted into digital signals. A nozzle system (e.g., a vibrating nozzle system) may be used to generate droplets (e.g., aqueous droplets) comprising individual cells and/or cellular constituents. Droplets including cells and/or cellular constituents of interest (e.g., as determined via optical detection) may be labeled with an electric charge (e.g., using an electrical charging ring), which charge may be used to separate such droplets from droplets including other cells and/or cellular constituents. For example, FACS may comprise labeling cells and/or cellular constituents with fluorescent markers (e.g., using internal and/or external biomarkers). Cells and/or cellular constituents may then be measured and identified one by one and sorted based on the emitted fluorescence of the marker or absence thereof. MACS may use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS may use microbubbles (e.g., glass microbubbles) labeled with antibodies to target cells of interest. Cells and/or cellular components coupled to microbubbles may float to a surface of a solution, thereby separating target cells and/or cellular components from other components of a sample. Cell separation techniques may be used to enrich for populations of cells of interest (e.g., prior to partitioning, as described herein). For example, a sample comprising a plurality of cells including a plurality of cells of a given type may be subjected to a positive separation process. The plurality of cells of the given type may be labeled with a fluorescent marker (e.g., based on an expressed cell surface marker or another marker) and subjected to a FACS process to separate these cells from other cells of the plurality of cells. The selected cells may then be subjected to subsequent partition-based analysis (e.g., as described herein) or other downstream analysis. The fluorescent marker may be removed prior to such analysis or may be retained. The fluorescent marker may comprise an identifying feature, such as a nucleic acid barcode sequence and/or unique molecular identifier.

In another example, a first sample comprising a first plurality of cells including a first plurality of cells of a given type (e.g., immune cells expressing a particular marker or combination of markers) and a second sample comprising a second plurality of cells including a second plurality of cells of the given type may be subjected to a positive separation process. The first and second samples may be collected from the same or different subjects, at the same or different types, from the same or different bodily locations or systems, using the same or different collection techniques. For example, the first sample may be from a first subject and the second sample may be from a second subject different than the first subject. The first plurality of cells of the first sample may be provided a first plurality of fluorescent markers configured to label the first plurality of cells of the given type. The second plurality of cells of the second sample may be provided a second plurality of fluorescent markers configured to label the second plurality of cells of the given type. The first plurality of fluorescent markers may include a first identifying feature, such as a first barcode, while the second plurality of fluorescent markers may include a second identifying feature, such as a second barcode, that is different than the first identifying feature. The first plurality of fluorescent markers and the second plurality of fluorescent markers may fluoresce at the same intensities and over the same range of wavelengths upon excitation with a same excitation source (e.g., light source, such as a laser). The first and second samples may then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples may undergo separate FACS processes and the positively selected cells of the given type from the first sample and the positively selected cells of the given type from the second sample may then be combined for subsequent analysis. The encoded identifying features of the different fluorescent markers may be used to identify cells originating from the first sample and cells originating from the second sample. For example, the first and second identifying features may be configured to interact (e.g., in partitions, as described herein) with nucleic acid barcode molecules (e.g., as described herein) to generate barcoded nucleic acid products detectable using, e.g., nucleic acid sequencing.

A sample may be a fixed sample. For example, a sample may comprise a plurality of fixed samples, such as a plurality of fixed cells or fixed nuclei. Alternatively, or in addition, a sample may comprise a fixed tissue. Fixation of cell or cellular constituent, or a tissue comprising a plurality of cells or nuclei, may comprise application of a chemical species or chemical stimulus. The term “fixed” as used herein with regard to biological samples generally refers to the state of being preserved from decay and/or degradation. “Fixation” generally refers to a process that results in a fixed sample, and in some instances can include contacting the biomolecules within a biological sample with a fixative (or fixation reagent) for some amount of time, whereby the fixative results in covalent bonding interactions such as crosslinks between biomolecules in the sample. A “fixed biological sample” may generally refer to a biological sample that has been contacted with a fixation reagent or fixative. For example, a formaldehyde-fixed biological sample has been contacted with the fixation reagent formaldehyde. “Fixed cells” or “fixed tissues” generally refer to cells or tissues that have been in contact with a fixative under conditions sufficient to allow or result in the formation of intra- and inter-molecular covalent crosslinks between biomolecules in the biological sample. Generally, contact of biological sample (e.g., a cell or nucleus) with a fixation reagent (e.g., paraformaldehyde or PFA) results in the formation of intra- and inter-molecular covalent crosslinks between biomolecules in the biological sample. In some cases, provision of the fixation reagent, such as formaldehyde, may result in covalent aminal crosslinks within RNA, DNA, and/or protein molecules. For example, the widely used fixative reagent, paraformaldehyde or PFA, fixes tissue samples by catalyzing crosslink formation between basic amino acids in proteins, such as lysine and glutamine. Both intra-molecular and inter-molecular crosslinks can form in the protein. These crosslinks can preserve protein secondary structure and also eliminate enzymatic activity in the preserved tissue sample. Examples of fixation reagents include but are not limited to aldehyde fixatives (e.g., formaldehyde, also commonly referred to as “paraformaldehyde,” “PFA,” and “formalin”; glutaraldehyde; etc.), imidoesters, NHS (N-Hydroxysuccinimide) esters, and the like.

Other examples of fixation reagents include, for example, organic solvents such as alcohols (e.g., methanol or ethanol), ketones (e.g., acetone), and aldehydes (e.g., paraformaldehyde, formaldehyde (e.g., formalin), or glutaraldehyde). As described herein, cross-linking agents may also be used for fixation including, without limitation, disuccinimidyl suberate (DSS), dimethylsuberimidate (DMS), formalin, and dimethyladipimidate (DMA), dithio-bis(-succinimidyl propionate) (DSP), disuccinimidyl tartrate (DST), and ethylene glycol bis(succinimidyl succinate) (EGS). In some cases, a cross-linking agent may be a cleavable cross-linking agent (e.g., thermally cleavable, photocleavable, etc.). In some cases, more than one fixation reagent can be used in combination when preparing a fixed biological sample. Changes to a characteristic or a set of characteristics of a cell or cellular constituents (e.g., incurred upon interaction with one or more fixation agents) may be at least partially reversible (e.g., via rehydration or de-crosslinking). Alternatively, changes to a characteristic or set of characteristics of a cell or cellular constituents may be intended to be non-reversible.

### Single Cell Workflows

In some aspects, provided herein are single cell circularization-based 3′/5′ approaches for generating a sequencing library (and methods for sequencing).

In some aspects, the single cell circularization-based dual 3′/5′ approach generates extended products where a 5′ portion or 5′ end of the nucleic acid analyte is proximal to a barcode region and a 3′ portion or 3′ end of the analyte is distal to the barcode region, and following circularization, a sequencing library can be generating with fragments including 3′ portion or 3′ end and the single cell barcode, and fragments including 5′ portion or 5′ end and the single cell barcode. In some embodiments, the method includes providing a plurality of partitions, wherein a partition of the plurality of partitions comprises: a nucleic acid analyte from the single cell, a primer molecule, wherein the primer molecule comprises a sequence that hybridizes to the nucleic acid analyte, and a nucleic acid barcode molecule, wherein the nucleic acid barcode molecule includes a single cell barcode sequence and a template switching oligonucleotide (TSO) comprising a hybridization region at its 3′ end. In some embodiments, the hybridization region of the TSO is a polyG sequence. In some embodiments, the method further includes extending the primer molecule using a reverse transcriptase having terminal transferase activity to generate a cDNA molecule comprising a sequence that is complementary to the hybridization region of the TSO. In some embodiments, the sequence that is complementary is a polyC sequence. In some embodiments, the method further includes hybridizing the cDNA molecule to the hybridization region of the TSO. In some embodiments, the method further includes extending the cDNA molecule using the nucleic acid barcode molecule as a template. In some embodiments, the method includes circularizing the cDNA molecule to generate a circularized cDNA molecule. In the circularizing includes ligating. In some embodiments, the ligating is Y-ligation, ligating a 5′ phosphate of the molecule to a 3′ hydroxy of the same molecule adjacent to a double-stranded region. In some embodiments, the ligating includes ligating a double-stranded region to an additional double-stranded single-molecule oligonucleotide, such as a hairpin. In some embodiments, the method includes sequencing (i) all or a part of a sequence of the cDNA molecule at its 5′ end, or a complement thereof, (ii) all or a part of a sequence of the cDNA molecule at its 3′ end, or a complement thereof, and (iii) the cell barcode sequence, or a complement thereof.

In some aspects, the single cell circularization-based dual 3′/5′ approach generates extended products where a 3′ portion or 3′ end of the nucleic acid analyte (e.g., a polyA tail for an mRNA analyte) is proximal to a barcode region and a 5′ portion or 5′ end of the analyte is distal to the barcode region, and following circularization, a sequencing library can be generating with fragments including 3′ portion or 3′ end and the single cell barcode, and fragments including 5′ portion or 5′ end and the single cell barcode. In some embodiments, providing a plurality of partitions, wherein a partition of the plurality of partitions comprises: a nucleic acid analyte from the single cell, a nucleic acid barcode molecule, wherein the nucleic acid barcode molecule comprises a cell barcode sequence and a hybridization region at its 3′ end. In some embodiments, the method further includes hybridizing the nucleic acid analyte to the hybridization region. In some embodiments, the method further includes extending the nucleic acid barcode molecule using the nucleic acid analyte as a template, thereby generating a cDNA molecule. In some embodiments, the method further includes circularizing the cDNA molecule to generate a circularized cDNA molecule. In some embodiments, the method further includes sequencing (i) all or a part of a sequence of the cDNA molecule at its 5′ end, or a complement thereof, (ii) all or a part of a sequence of the cDNA molecule at its 3′ end, or a complement thereof, and (iii) the cell barcode sequence, or a complement thereof.

In some embodiments, all or a part of a sequence of the cDNA molecule at its 5′ end includes about 50, about 75, about 100, about 150, about 200, about 250, about 300 or more nucleotides, and/or all or a part of a sequence of the cDNA molecule at its 3′ end comprises about 50, about 75, about 100, about 150, about 200, about 250, about 300, about 350, about 400, about 450, about 500 or more nucleotides.

In some embodiments, circularizing the cDNA molecule utilizes a ligase (e.g., a PBCV-1 DNA ligase, a Chlorella virus DNA ligase, a single stranded DNA ligase, or a T4 DNA ligase).

In some embodiments, the circularized cDNA molecule comprises one or more cell barcodes, wherein the one or more cell barcodes comprises the cell barcode sequence. In some embodiments, the circularized cDNA molecule comprises one or more functional domains, one or more unique molecule identifiers (UMIs), or combinations thereof.

In some embodiments, the one or more functional domains comprise one or more primer sites. The one or more primer sites may be used to amplify the cDNA molecule both at its 5′ end and at its 3′ end, thereby generating a plurality of nucleic acid analyte amplicons.

In some embodiments, the plurality of nucleic acid analyte amplicons include: a 5′ nucleic acid analyte amplicon including sequences of (i) all or part of the sequence of the cDNA molecule at its 5′ end, or a complement thereof, (ii) one or more UMIs, or complements thereof, and (iii) one or more spatial barcodes, or complements thereof; and a 3′ nucleic acid analyte amplicon comprising sequences of (i) all or part of the sequence of the cDNA molecule at its 3′ end, or a complement thereof, (ii) one or more UMIs, or complements thereof, and (iii) one or more spatial barcodes, or complements thereof.

In some embodiments, the plurality of nucleic acid analyte amplicons include: a 5′ nucleic acid analyte amplicon comprising sequences of (i) all or part of the sequence of the cDNA molecule at its 5′ end, or a complement thereof, (ii) one UMI, or a complement thereof, and (iii) the spatial barcodes, or a complement thereof; and a 3′ nucleic acid analyte amplicon comprising sequences of (i) all or part of the sequence of the cDNA molecule at its 3′ end, or a complement thereof, (ii) one UMI, or a complement thereof, and (iii) the spatial barcodes, or a complement thereof.

### Systems and Methods for Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion or a well. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more beads. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead.

Unique identifiers, such as barcodes, may be injected into the droplets previous to, subsequent to, or concurrently with droplet generation, such as via a bead, as described elsewhere herein.

The methods and systems of the present disclosure may comprise methods and systems for generating one or more partitions such as droplets. The droplets may comprise a plurality of droplets in an emulsion. In some examples, the droplets may comprise droplets in a colloid. In some cases, the emulsion may comprise a microemulsion or a nanoemulsion. In some examples, the droplets may be generated with aid of a microfluidic device and/or by subjecting a mixture of immiscible phases to agitation (e.g., in a container). In some cases, a combination of the mentioned methods may be used for droplet and/or emulsion formation.

The partitions described herein may comprise small volumes, for example, less than about 10 microliters (μL), 5 μL, 1 μL, 10 nanoliters (nL), 5 nL, 1 nL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet-based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with beads, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Patent Application No. PCT/US2020/17785 and U.S. Patent Application Publication No. US20220025438, which are entirely incorporated herein by reference for all purposes.

### Microfluidic Systems

Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particles in partitions, methods of increasing the throughput of droplet generation, and various geometries, architectures, and configurations of microfluidic devices and channels are described in U.S. Patent Application Publication Nos. 2019/0367997 and 2019/0064173, each of which is entirely incorporated herein by reference for all purposes.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as biological particles, in some cases, individual biological particles such as single cells. In some examples, reagents may be encapsulated and/or partitioned (e.g., co-partitioned with biological particles) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

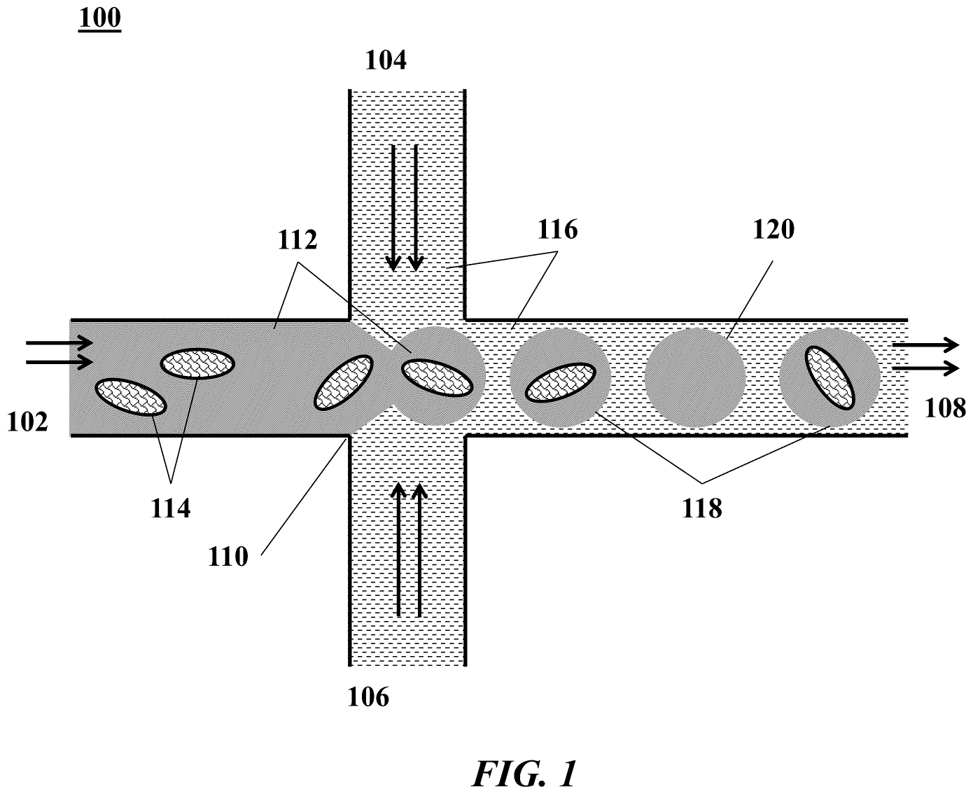

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, flows can be controlled so as to present a non-Poissonian distribution of single-occupied partitions while providing lower levels of unoccupied partitions (e.g., no more than about 50%, about 25%, or about 10% unoccupied). The above noted ranges of unoccupied partitions can be achieved while still providing any of the single occupancy rates described above.

As will be appreciated, the above-described occupancy rates are also applicable to partitions that include both biological particles and additional reagents, such as beads (e.g., gel beads) carrying nucleic acid barcode molecules (e.g., oligonucleotides).

In some examples, a partition of the plurality of partitions may comprise a single biological particle (e.g., a single cell or a single nucleus of a cell). In some examples, a partition of the plurality of partitions may comprise multiple biological particles. Such partitions may be referred to as multiply occupied partitions, and may comprise, for example, two, three, four or more cells and/or beads (e.g., beads) comprising nucleic acid barcode molecules within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

Microfluidic systems for partitioning are further described in U.S. Patent Application Pub. No. US 2015/0376609, which is hereby incorporated by reference in its entirety.

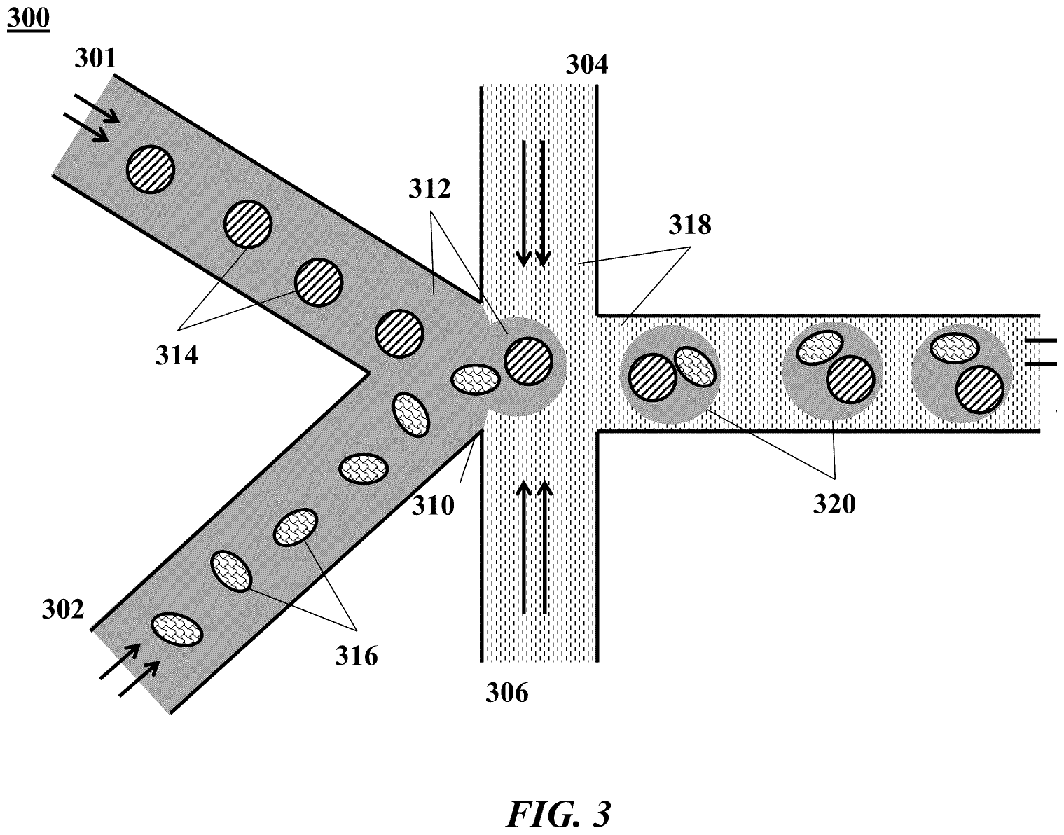

FIG. 3 shows an example of a microfluidic channel structure 300 for delivering barcode carrying beads to droplets. The channel structure 300 can include channel segments 301, 302, 304, 306 and 308 communicating at a channel junction 310. In operation, the channel segment 301 may transport an aqueous fluid 312 that includes a plurality of beads 314 (e.g., with nucleic acid molecules, e.g., nucleic acid barcode molecules or barcoded oligonucleotides, molecular tags) along the channel segment 301 into junction 310. The plurality of beads 314 may be sourced from a suspension of beads. For example, the channel segment 301 may be connected to a reservoir comprising an aqueous suspension of beads 314. The channel segment 302 may transport the aqueous fluid 312 that includes a plurality of biological particles 316 along the channel segment 302 into junction 310. The plurality of biological particles 316 may be sourced from a suspension of biological particles. For example, the channel segment 302 may be connected to a reservoir comprising an aqueous suspension of biological particles 316. In some instances, the aqueous fluid 312 in either the first channel segment 301 or the second channel segment 302, or in both segments, can include one or more reagents, as further described below. A second fluid 318 that is immiscible with the aqueous fluid 312 (e.g., oil) can be delivered to the junction 310 from each of channel segments 304 and 306. Upon meeting of the aqueous fluid 312 from each of channel segments 301 and 302 and the second fluid 318 from each of channel segments 304 and 306 at the channel junction 310, the aqueous fluid 312 can be partitioned as discrete droplets 320 in the second fluid 318 and flow away from the junction 310 along channel segment 308. The channel segment 308 may deliver the discrete droplets to an outlet reservoir fluidly coupled to the channel segment 308, where they may be harvested. As an alternative, the channel segments 301 and 302 may meet at another junction upstream of the junction 310. At such junction, beads and biological particles may form a mixture that is directed along another channel to the junction 310 to yield droplets 320. The mixture may provide the beads and biological particles in an alternating fashion, such that, for example, a droplet comprises a single bead and a single biological particle.

### Controlled Partitioning

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

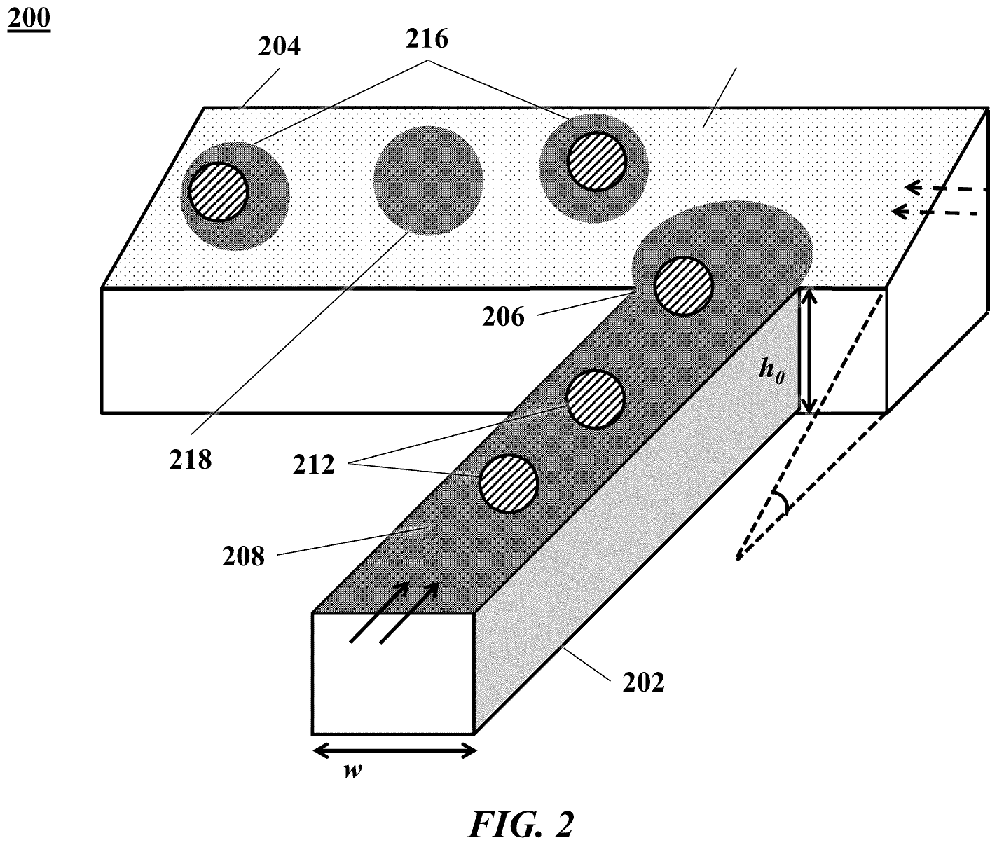

FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 may be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, h0, α, etc.) of the channel structure 200. A plurality of droplets can be collected in the reservoir 204 by continuously injecting the aqueous fluid 208 from the channel segment 202 through the junction 206.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 may be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 208 in the channel segment 202 can comprise biological particles. In some instances, the aqueous fluid 208 can have a substantially uniform concentration or frequency of biological particles. As with the beads, the biological particles can be introduced into the channel segment 202 from a separate channel. The frequency or concentration of the biological particles in the aqueous fluid 208 in the channel segment 202 may be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads may be upstream or downstream of the second separate channel introducing the biological particles.

The second fluid 210 can comprise an oil, such as a fluorinated oil, that includes a fluorosurfactant for stabilizing the resulting droplets, for example, inhibiting subsequent coalescence of the resulting droplets.

In some instances, the second fluid 210 may not be subjected to and/or directed to any flow in or out of the reservoir 204. For example, the second fluid 210 may be substantially stationary in the reservoir 204. In some instances, the second fluid 210 may be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 may be subjected and/or directed to flow in or out of the reservoir 204. For example, the reservoir 204 can be a channel directing the second fluid 210 from upstream to downstream, transporting the generated droplets.

Systems and methods for controlled partitioning are described further in International Patent Application No. PCT/US2018/047551 and U.S. Patent Application Publication No. US2020/0290048, which are hereby incorporated by reference in their entirety.

### Cell Beads

In another aspect, in addition to or as an alternative to droplet-based partitioning, biological particles (e.g., cells) may be comprised within (e.g., encapsulated within) a particulate material to form a “cell bead”. Methods and compositions drawn to cell beads and the like are described further in International Patent Application No. PCT/US2018/016019 and U.S. Patent Application No. US2018/0216162, which are hereby incorporated by reference in their entirety.

A cell bead can contain a biological particle (e.g., a cell) or macromolecular constituents (e.g., RNA, DNA, proteins, etc.) of a biological particle. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

Cell beads can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli (or reagents).

Suitable polymers or gels may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. The conditions sufficient to polymerize or gel the precursors may comprise any conditions sufficient to polymerize or gel the precursors. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), electromagnetic radiation, mechanical stimuli, or any combination thereof.

In some cases, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 1, may be readily used in encapsulating biological particles (e.g., cells) as described herein. Exemplary methods for encapsulating biological particles (e.g., cells) are also further described in U.S. Patent Application Pub. No. US 2015/0376609 and International Patent Application No. PCT/US2018/016019, which are hereby incorporated by reference in their entirety. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the bead that includes the entrained biological particles. Examples of polymer precursor/initiator pairs include those described in U.S. Patent Application Publication No. 2014/0378345, which is entirely incorporated herein by reference for all purposes.

In some cases, encapsulated biological particles can be selectively releasable from the cell bead, such as through passage of time or upon application of a particular stimulus, that degrades the bead sufficiently to allow the biological particles (e.g., cell), or its other contents to be released from the bead, such as into a partition (e.g., droplet). Exemplary stimuli suitable for degradation of the bead are described in U.S. Patent Application Publication No. 2014/0378345, which is entirely incorporated herein by reference for all purposes.

The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The encapsulation of biological particles may constitute the partitioning of the biological particles into which other reagents are co-partitioned. Alternatively, or in addition, encapsulated biological particles may be readily deposited into other partitions (e.g., droplets) as described above.

### Beads

Nucleic acid barcode molecules may be delivered to a partition (e.g., a droplet or well) via a solid support or carrier (e.g., a bead). In some cases, nucleic acid barcode molecules are initially associated with the solid support and then released from the solid support upon application of a stimulus, which allows the nucleic acid barcode molecules to dissociate or to be released from the solid support. In specific examples, nucleic acid barcode molecules are initially associated with the solid support (e.g., bead) and then released from the solid support upon application of a biological stimulus, a chemical stimulus, a thermal stimulus, an electrical stimulus, a magnetic stimulus, and/or a photo stimulus.

The solid support may be a bead. A solid support, e.g., a bead, may be porous, non-porous, hollow, solid, semi-solid, and/or a combination thereof. Beads may be solid, semi-solid, semi-fluidic, fluidic, and/or a combination thereof. In some instances, a solid support, e.g., a bead, may be at least partially dissolvable, disruptable, and/or degradable. In some cases, a solid support, e.g., a bead, may not be degradable. In some cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In some cases, the solid support, e.g., the bead, may be a silica bead. In some cases, the solid support, e.g., a bead, can be rigid. In other cases, the solid support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., via a nucleic acid barcode molecule), to a partition via any suitable mechanism. Nucleic acid barcode molecules can be delivered to a partition via a bead. Beads are described in further detail below.

In some cases, nucleic acid barcode molecules can be initially associated with the bead and then released from the bead. Release of the nucleic acid barcode molecules can be passive (e.g., by diffusion out of the bead). In addition, or alternatively, release from the bead can be upon application of a stimulus which allows the nucleic acid barcode molecules to dissociate or to be released from the bead. Such stimulus may disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

Methods and systems for partitioning barcode carrying beads into droplets are provided herein, and in in US. Patent Publication Nos. 2019/0367997 and 2019/0064173, and International Patent Application No. PCT/US20/17785, each of which is herein entirely incorporated by reference for all purposes.

A bead may be porous, non-porous, solid, semi-solid, semi-fluidic, fluidic, and/or a combination thereof. In some instances, a bead may be dissolvable, disruptable, and/or degradable. Degradable beads, as well as methods for degrading beads, are described in International Patent Application Publication No. PCT/US2014/044398, which is hereby incorporated by reference in its entirety. In some cases, any combination of stimuli, e.g., stimuli described in PCT/US2014/044398 and US Patent Application Pub. No. 2015/0376609, hereby incorporated by reference in its entirety, may trigger degradation of a bead. For example, a change in pH may enable a chemical agent (e.g., DTT) to become an effective reducing agent. In other examples, a reducing agent (e.g., DTT) may be used to degrade the bead.

In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. See, e.g., PCT/US2014/044398, which is hereby incorporated by reference in its entirety. Beads may also be formed from materials other than polymers, including lipids, micelles, ceramics, glass-ceramics, material composites, metals, other inorganic materials, and others.

In some cases, the bead may comprise covalent or ionic bonds between polymeric precursors (e.g., monomers, oligomers, linear polymers), nucleic acid barcode molecules (e.g., oligonucleotides), primers, and other entities. In some cases, the covalent bonds can be carbon-carbon bonds, thioether bonds, or carbon-heteroatom bonds.

In some cases, a plurality of nucleic acid barcode molecules may be attached to a bead. The nucleic acid barcode molecules may be attached directly or indirectly to the bead. In some cases, the nucleic acid barcode molecules may be covalently linked to the bead. In some cases, the nucleic acid barcode molecules are covalently linked to the bead via a linker. In some cases, the linker is a degradable linker. In some cases, the linker comprises a labile bond configured to release the nucleic acid barcode molecule of said plurality of nucleic acid barcode molecules. In some cases, the labile bond comprises a disulfide linkage.

Activation or disruption of disulfide linkages within a bead can be controlled such that only a small number of disulfide linkages are activated or disrupted. Methods of controlling activation of disulfide linkages within a bead are described in International Patent Application No. PCT/US2014/044398, which is hereby incorporated by reference in its entirety.

In some cases, a bead may comprise an acrydite moiety, which in certain aspects may be used to attach one or more nucleic acid barcode molecules (e.g., barcode sequence, nucleic acid barcode molecule, barcoded oligonucleotide, primer, or other oligonucleotide) to the bead. Acrydite moieties, as well as their uses in attaching nucleic acid molecules to beads, are described in PCT/US2014/044398, which is hereby incorporated by reference in its entirety.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule, e.g., a nucleic acid barcode molecule described herein.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. Exemplary precursors comprising functional groups are described in PCT/US2014/044398, which is hereby incorporated by reference in its entirety.

Other non-limiting examples of labile bonds that may be coupled to a precursor or bead are described in PCT/US2014/044398, which is hereby incorporated by reference in its entirety. A bond may be cleavable via other nucleic acid molecule targeting enzymes, such as restriction enzymes (e.g., restriction endonucleases), as described further below.

In some cases, a plurality of nucleic acid barcode molecules may be attached to a bead via non-covalent bonds. For example, the plurality of nucleic acid barcode molecules may be associated with a bead via an ionic interaction, electrostatic interactions, metallic bond, hydrogen bonding, van der Waals interactions, etc. In some cases, the non-covalent bond may be degraded upon application of a stimulus, e.g., a thermal, photo, magnetic, electrical, chemical stimulus (e.g., change in pH, ion concentration, etc.).

Species may be encapsulated in beads during bead generation (e.g., during polymerization of precursors). Such species may or may not participate in polymerization. See, e.g., PCT/US2014/044398, which is hereby incorporated by reference in its entirety. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Alternatively, or in addition, species may be partitioned in a partition (e.g., droplet) during or subsequent to partition formation. Such species may include, without limitation, the abovementioned species that may also be encapsulated in a bead.

In some cases, beads can be non-covalently loaded with and/or coupled to one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

### Nucleic Acid Barcode Molecules for Single Cell Workflows

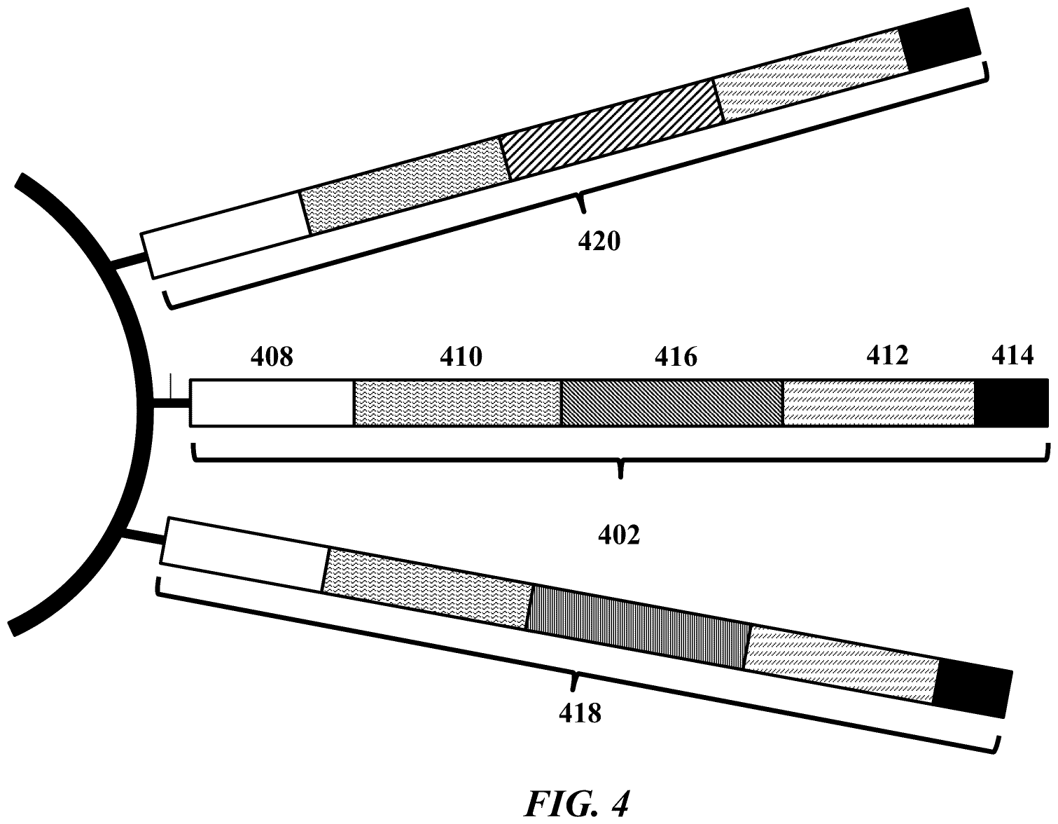

FIG. 4 illustrates an example of a barcode carrying bead. A nucleic acid barcode molecule 402 can be coupled to a bead 404 by a releasable linkage 406, such as, for example, a disulfide linker. The same bead 404 may be coupled (e.g., via releasable linkage) to one or more other nucleic acid barcode molecules 418, 420. The nucleic acid barcode molecule 402 may be or comprise a barcode. As noted elsewhere herein, the structure of the barcode may comprise a number of sequence elements. The nucleic acid barcode molecule 402 may comprise a functional sequence 408 that may be used in subsequent processing. For example, the functional sequence 408 may include one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof, and a region that is complementary to a region of an additional oligonucleotide used for generating an extended product for circularization. The nucleic acid barcode molecule 402 may comprise a barcode sequence 410 for use in barcoding the sample (e.g., DNA, RNA, protein, etc.). In some cases, the barcode sequence 410 can be bead-specific such that the barcode sequence 410 is common to all nucleic acid barcode molecules (e.g., including nucleic acid barcode molecule 402) coupled to the same bead 404. Alternatively, or in addition, the barcode sequence 410 can be partition-specific such that the barcode sequence 410 is common to all nucleic acid barcode molecules coupled to one or more beads that are partitioned into the same partition. The nucleic acid barcode molecule 402 may comprise sequence 412 complementary to an analyte of interest, e.g., a priming sequence. Sequence 412 can be a poly-T sequence complementary to a poly-A tail of an mRNA analyte, a targeted priming sequence, and/or a random priming sequence. The nucleic acid barcode molecule 402 may comprise an anchoring sequence 414 to ensure that the specific priming sequence 412 hybridizes at the sequence end (e.g., of the mRNA). For example, the anchoring sequence 414 can include a random short sequence of nucleotides, such as a 1-mer, 2-mer, 3-mer or longer sequence, which can ensure that a poly-T segment is more likely to hybridize at the sequence end of the poly-A tail of the mRNA. In some embodiments, the nucleic acid barcode molecule includes two barcode sequences separated by a primer region (including one or more primer binding sites).

The nucleic acid barcode molecule 402 may comprise a unique molecular identifying sequence 416 (e.g., unique molecular identifier (UMI)). In some cases, the unique molecular identifying sequence 416 may comprise from about 5 to about 8 nucleotides. Alternatively, the unique molecular identifying sequence 416 may compress less than about 5 or more than about 8 nucleotides. The unique molecular identifying sequence 416 may be a unique sequence that varies across individual nucleic acid barcode molecules (e.g., 402, 418, 420, etc.) coupled to a single bead (e.g., bead 404). In some cases, the unique molecular identifying sequence 416 may be a random sequence (e.g., such as a random N-mer sequence). For example, the UMI may provide a unique identifier of the starting analyte (e.g., mRNA) molecule that was captured, in order to allow quantitation of the number of original expressed RNA molecules. As will be appreciated, although FIG. 4 shows three nucleic acid barcode molecules 402, 418, 420 coupled to the surface of the bead 404, an individual bead may be coupled to any number of individual nucleic acid barcode molecules, for example, from one to tens to hundreds of thousands, millions, or even a billion of individual nucleic acid barcode molecules. The respective barcodes for the individual nucleic acid barcode molecules can comprise both common sequence segments or relatively common sequence segments (e.g., 408, 410, 412, etc.) and variable or unique sequence segments (e.g., 416) between different individual nucleic acid barcode molecules coupled to the same bead.

In operation, a biological particle (e.g., cell, DNA, RNA, etc.) can be co-partitioned along with a barcode bearing bead 404. The nucleic acid barcode molecules 402, 418, 420 can be released from the bead 404 in the partition. By way of example, in the context of analyzing sample RNA, the poly-T segment (e.g., 412) of one of the released nucleic acid barcode molecules (e.g., 402) can hybridize to the poly-A tail of a mRNA molecule. Reverse transcription may result in a cDNA transcript of the mRNA, but which transcript includes each of the sequence segments 408, 410, 416 of the nucleic acid barcode molecule 402. Because the nucleic acid barcode molecule 402 comprises an anchoring sequence 414, it will more likely hybridize to and prime reverse transcription at the sequence end of the poly-A tail of the mRNA. Within any given partition, all of the cDNA transcripts of the individual mRNA molecules may include a common barcode sequence segment 410. However, the transcripts made from the different mRNA molecules within a given partition may vary at the unique molecular identifying sequence 412 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid barcode molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise at least 2, 3, 4, 5, 6, 7, 8, 9, 10 or more different capture sequences or binding sequences configured to bind to different respective capture sequences or binding sequences, respectively. Alternatively, or in addition, a bead may comprise at most about 10, 9, 8, 7, 6, 5, 4, 3, or 2 different capture sequences or binding sequences configured to bind to different respective capture sequences or binding sequences. In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, an biological particle (e.g., cell, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules.

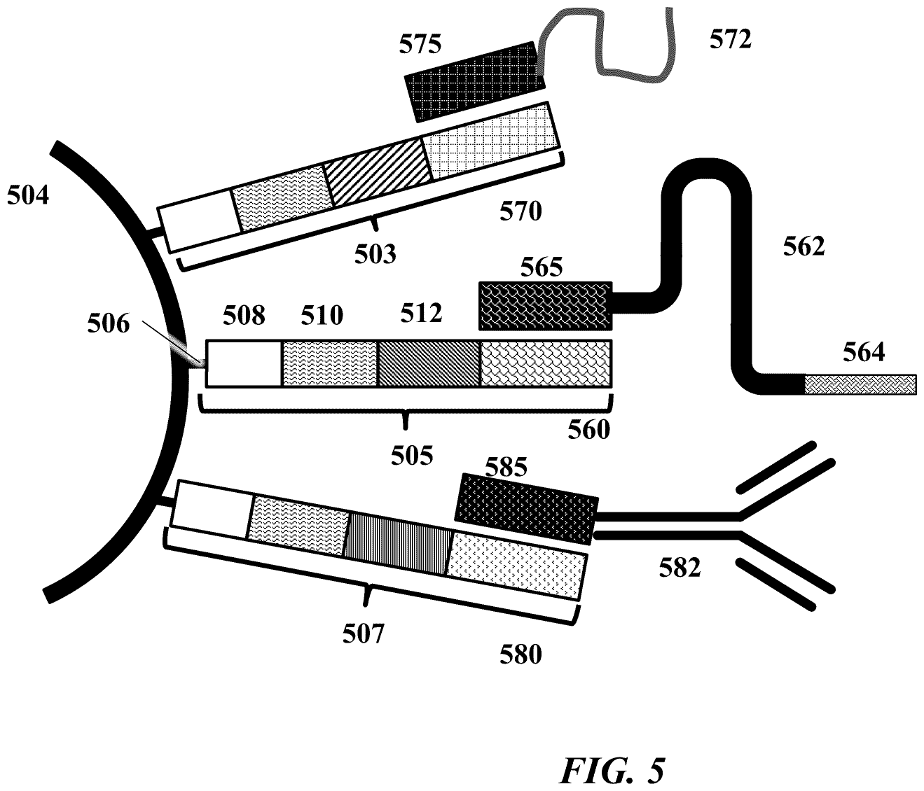

FIG. 5 illustrates another example of a barcode carrying bead. A nucleic acid barcode molecule 505, such as an oligonucleotide, can be coupled to a bead 504 by a releasable linkage 506, such as, for example, a disulfide linker. The nucleic acid barcode molecule 505 may comprise a first capture sequence 560. The same bead 504 may be coupled (e.g., via releasable linkage) to one or more other nucleic acid molecules 503, 507 comprising other capture sequences. The nucleic acid barcode molecule 505 may be or comprise a barcode. As noted elsewhere herein, the structure of the barcode may comprise a number of sequence elements, such as a functional sequence 508 (e.g., flow cell attachment sequence, sequencing primer sequence, region of complementarity with an additional oligonucleotide used for generating an extension product, etc.), a barcode sequence 510 (e.g., bead-specific sequence common to bead, partition-specific sequence common to partition, etc.), and a unique molecular identifier 512 (e.g., unique sequence within different molecules attached to the bead), or partial sequences thereof. The capture sequence 560 may be configured to attach to a corresponding capture sequence 565. In some instances, the corresponding capture sequence 565 may be coupled to another molecule that may be an analyte or an intermediary carrier. For example, as illustrated in FIG. 5, the corresponding capture sequence 565 is coupled to a guide RNA molecule 562 comprising a target sequence 564, wherein the target sequence 564 is configured to attach to the analyte. Another oligonucleotide molecule 507 attached to the bead 504 comprises a second capture sequence 580 which is configured to attach to a second corresponding capture sequence 585. As illustrated in FIG. 5, the second corresponding capture sequence 585 is coupled to an antibody 582. In some cases, the antibody 582 may have binding specificity to an analyte (e.g., surface protein). Alternatively, the antibody 582 may not have binding specificity. Another oligonucleotide molecule 503 attached to the bead 504 comprises a third capture sequence 570 which is configured to attach to a third corresponding capture sequence 575. As illustrated in FIG. 5, the third corresponding capture sequence 575 is coupled to a molecule 572. The molecule 572 may or may not be configured to target an analyte. The other oligonucleotide molecules 503, 507 may comprise the other sequences (e.g., functional sequence, barcode sequence, UMI, etc.) described with respect to oligonucleotide molecule 505. While a single oligonucleotide molecule comprising each capture sequence is illustrated in FIG. 5, it will be appreciated that, for each capture sequence, the bead may comprise a set of one or more oligonucleotide molecules each comprising the capture sequence. For example, the bead may comprise any number of sets of one or more different capture sequences. Alternatively, or in addition, the bead 504 may comprise other capture sequences. Alternatively, or in addition, the bead 504 may comprise fewer types of capture sequences (e.g., two capture sequences). Alternatively, or in addition, the bead 504 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression. In some embodiments, two barcode sequences of the nucleic acid barcode molecule are separated by a primer region (e.g., including at least one priming sequence).

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. See, e.g., PCT/US2014/044398, which is hereby incorporated by reference in its entirety.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. See, e.g., PCT/US2014/044398, which is hereby incorporated by reference in its entirety.

As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences (e.g., restriction sites, transposition sites). Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, beads are provided that include large numbers of the above described nucleic acid barcode molecules releasably attached to the beads, where all or at least a subset of the nucleic acid barcode molecules attached to a particular bead will include a common nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid barcode molecules into the partitions, as they are capable of carrying large numbers of nucleic acid barcode molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, beads can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. In some embodiments, the number of nucleic acid molecules including the barcode sequence on an individual bead is between about 1,000 to about 10,000 nucleic acid molecules, about 5,000 to about 50,000 nucleic acid molecules, about 10,000 to about 100,000 nucleic acid molecules, about 50,000 to about 1,000,000 nucleic acid molecules, about 100,000 to about 10,000,000 nucleic acid molecules, about 1,000,000 to about 1 billion nucleic acid molecules.

Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acids, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules.

In some cases, the resulting population of partitions provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences. Additionally, each partition of the population can include between about 1,000 to about 10,000 nucleic acid barcode molecules, about 5,000 to about 50,000 nucleic acid barcode molecules, about 10,000 to about 100,000 nucleic acid barcode molecules, about 50,000 to about 1,000,000 nucleic acid barcode molecules, about 100,000 to about 10,000,000 nucleic acid barcode molecules, about 1,000,000 to about 1 billion nucleic acid barcode molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles and may be degraded for release of the attached nucleic acid molecules through exposure to a reducing agent, such as DTT.

### Single Cell Workflow Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

The methods and systems of the present disclosure may comprise microfluidic devices and methods of use thereof, which may be used for co-partitioning biological particles with reagents. Such systems and methods are described in U.S. Patent Publication No. US/20190367997, which is herein incorporated by reference in its entirety for all purposes.

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments of the microfluidic devices described elsewhere herein may be coupled to any of a variety of different fluid sources or receiving components, including reservoirs, tubing, manifolds, or fluidic components of other systems. As will be appreciated, the microfluidic channel structures may have various geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion-based partitioning such as encapsulation of biological particles that may be in addition to or in place of droplet partitioning, where any pore size of the encapsulate is sufficiently small to retain nucleic acid fragments of a given size, following cellular disruption.

Alternatively, or in addition to, the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., a cell or a nucleus in a polymer matrix), the biological particles may be exposed to an appropriate stimulus to release the biological particles or their contents from a co-partitioned bead. For example, in some cases, a chemical stimulus may be co-partitioned along with an encapsulated biological particle to allow for the degradation of the bead and release of the cell or its contents into the larger partition. In some cases, this stimulus may be the same as the stimulus described elsewhere herein for release of nucleic acid molecules (e.g., oligonucleotides) from their respective bead. In alternative examples, this may be a different and non-overlapping stimulus, in order to allow an encapsulated biological particle to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

Additional reagents may also be co-partitioned with the biological particle, such as endonucleases to fragment an biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, restriction enzymes, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some cases, template switching can be used to increase the length of a cDNA. In some cases, template switching can be used to append a predefined nucleic acid sequence to the cDNA. Template switching is further described in International Patent Application No. PCT/US2017/068320 and U.S. Pat. No. 10,011,872, which are hereby incorporated by reference in their entirety. Template switching oligonucleotides may comprise a hybridization region and a template region. Template switching oligonucleotides are further described in PCT/US2017/068320 and U.S. Pat. No. 10,011,872 which are hereby incorporated by reference in their entirety.

Any of the reagents described in this disclosure may be encapsulated in, or otherwise coupled to, a droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, and oligonucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles. In some aspects, this is performed by co-partitioning the individual biological particle or groups of biological particles with the unique identifiers, such as described above (with reference to FIG. 1 or 2).

In some cases, additional beads can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of beads from each source, while ensuring a given pairing or combination of such beads into a partition with a given number of biological particles (e.g., one biological particle and one bead per partition).

In some embodiments, following the generation of barcoded nucleic acid molecules according to methods disclosed herein, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken, and the contents of the droplet pooled for additional operations.

### Wells

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). In some embodiments, a well of a fluidic device is fluidically connected to another well of the fluidic device. Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

A well may comprise free reagents and/or reagents encapsulated in, or otherwise coupled to or associated with, beads, beads, or droplets.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In some cases, a well comprises a bead, or droplet that comprises a set of reagents that has a similar attribute (e.g., a set of enzymes, a set of minerals, a set of oligonucleotides, a mixture of different barcode molecules, a mixture of identical barcode molecules). In other cases, a bead or droplet comprises a heterogeneous mixture of reagents. In some cases, the heterogeneous mixture of reagents can comprise all components necessary to perform a reaction. In some cases, such mixture can comprise all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

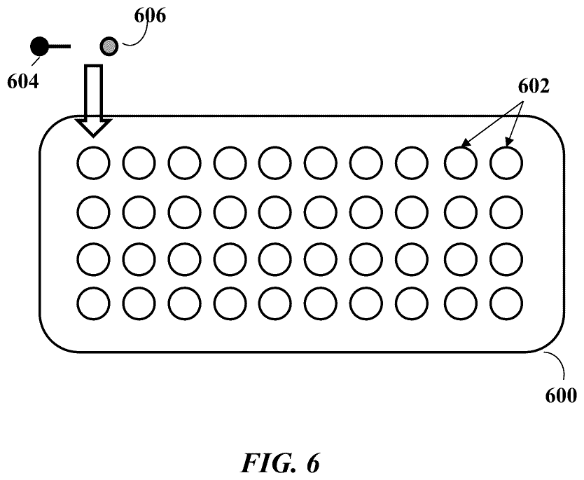

FIG. 6 schematically illustrates an example of a microwell array. The array can be contained within a substrate 600. The substrate 600 comprises a plurality of wells 602. The wells 602 may be of any size or shape, and the spacing between the wells, the number of wells per substrate, as well as the density of the wells on the substrate 600 can be modified, depending on the particular application. In one such example application, a sample molecule 606, which may comprise a cell or cellular components (e.g., nucleic acid molecules) is co-partitioned with a bead 604, which may comprise a nucleic acid barcode molecule coupled thereto. The wells 602 may be loaded using gravity or other loading technique (e.g., centrifugation, liquid handler, acoustic loading, optoelectronic, etc.). In some instances, at least one of the wells 602 contains a single sample molecule 606 (e.g., cell) and a single bead 604.

Reagents may be loaded into a well either sequentially or concurrently. In some cases, reagents are introduced to the device either before or after a particular operation. In some cases, reagents (which may be provided, in certain instances, in droplets, or beads) are introduced sequentially such that different reactions or operations occur at different steps. The reagents (or droplets, or beads) may also be loaded at operations interspersed with a reaction or operation step. For example, beads (or droplets) comprising reagents for fragmenting polynucleotides (e.g., restriction enzymes) and/or other enzymes (e.g., transposases, ligases, polymerases, etc.) may be loaded into the well or plurality of wells, followed by loading of droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead, or droplet. These beads, or droplets may be loaded into a partition (e.g., a microwell) before, after, or concurrently with the loading of a cell, such that each cell is contacted with a different bead, or droplet. This technique may be used to attach a unique nucleic acid barcode molecule to nucleic acid molecules obtained from each cell. Alternatively, or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in some instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or bead may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecules may be attached to a droplet or bead that has been partitioned into the well. The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. In some instances, the nucleic acid barcode molecules may be releasable from the bead or droplet. For instance, the nucleic acid barcode molecules may comprise a chemical cross-linker which may be cleaved upon application of a stimulus (e.g., photo-, magnetic, chemical, biological, stimulus). The nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In some instances nucleic acid barcode molecules attached to a bead in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to dynamic live-cell tracking, cell-cell interactions (when two or more cells are co-partitioned), cell proliferation, etc. Alternatively, or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

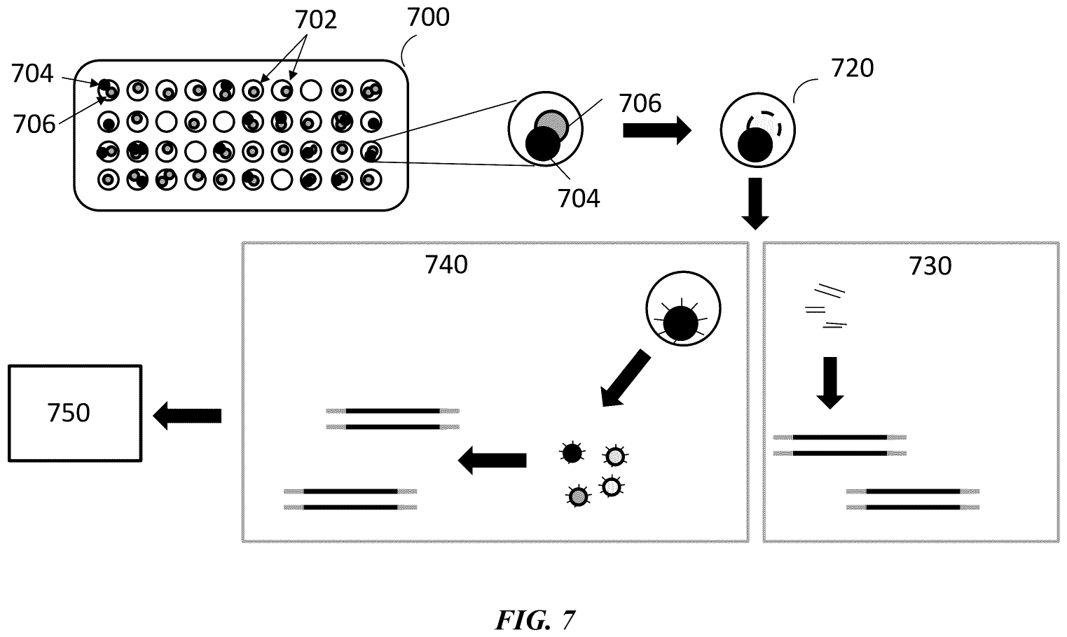

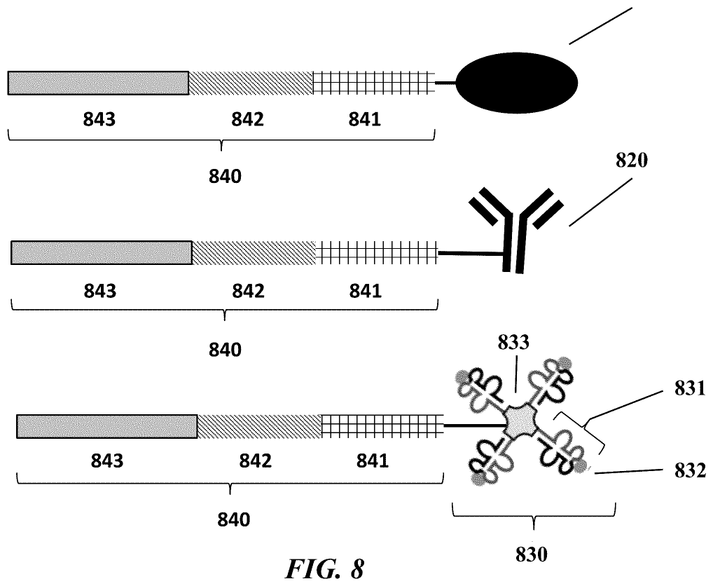

FIG. 7 schematically shows an example workflow for processing nucleic acid molecules within a sample. A substrate 700 comprising a plurality of microwells 702 may be provided. A sample 706 which may comprise a cell, cell bead, cellular components or analytes (e.g., proteins and/or nucleic acid molecules) can be co-partitioned, in a plurality of microwells 702, with a plurality of beads 704 comprising nucleic acid barcode molecules. During process 710, the sample 706 may be processed within the partition. For instance, in the case of live cells, the cell may be subjected to conditions sufficient to lyse the cells and release the analytes contained therein. In process 720, the bead 704 may be further processed

In some embodiments, the bead comprises nucleic acid barcode molecules that are attached thereto, and sample nucleic acid molecules (e.g., RNA, DNA) may attach, e.g., via hybridization, to the nucleic acid barcode molecules. Such attachment may occur on the bead. In process 730, the beads 704 from multiple wells 702 may be collected and pooled. Further processing may be performed in process 740. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 750, further characterization, such as sequencing may be performed to generate sequencing reads. The sequencing reads may yield information on individual cells or populations of cells, which may be represented visually or graphically, e.g., in a plot.

In some embodiments, the bead comprises nucleic acid barcode molecules that are releasably attached thereto, as described below. The bead may degrade or otherwise release the nucleic acid barcode molecules into the well 702; the nucleic acid barcode molecules may then be used to barcode nucleic acid molecules within the well 702. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 750, further characterization, such as sequencing may be performed to generate sequencing reads. The sequencing reads may yield information on individual cells or populations of cells, which may be represented visually or graphically, e.g., in a plot.

### Targeting GEX

The methods provided herein may comprise the use of a targeting process to, e.g., enrich selected nucleic acid molecules within a sample.

An exemplary target enrichment method may comprise providing a plurality of barcoded nucleic acid molecules and hybridizing barcoded nucleic acid molecules comprising targeted regions of interest to oligonucleotide probes (“baits”) which are complementary to the targeted regions of interest (or to regions near or adjacent to the targeted regions of interest). Baits may be attached to a capture molecule, including without limitation a biotin molecule. The capture molecule (e.g., biotin) can be used to selectively pull down the targeted regions of interest (for example, with magnetic streptavidin beads) to thereby enrich the resultant population of barcoded nucleic acid molecules for those containing the targeted regions of interest.

Another exemplary enrichment method may comprise providing a plurality of barcoded nucleic acid molecules comprising a plurality of different barcode sequences, identifying a barcode sequence of the plurality of different barcode sequences, and enriching barcoded nucleic acid molecules comprising the barcode sequence. Enriching may comprise performing a nucleic acid extension reaction using a barcoded nucleic acid molecule comprising the barcode sequence and a primer comprising a sequence specific for the barcode sequence to generate an enriched plurality of barcoded nucleic acid molecules comprising the barcode sequence of interest. Details of such processes and additional schemes are included in, for example, International Patent Application No. PCT/US2020/012413, U.S. Patent Application Publication No. US2022/0025435, and U.S. Pat. No. 11,000,049, and herein entirely incorporated by reference for all purposes.

The present disclosure provides methods and systems for multiplexing, and otherwise increasing throughput in, analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cell features may be used to characterize biological particles and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

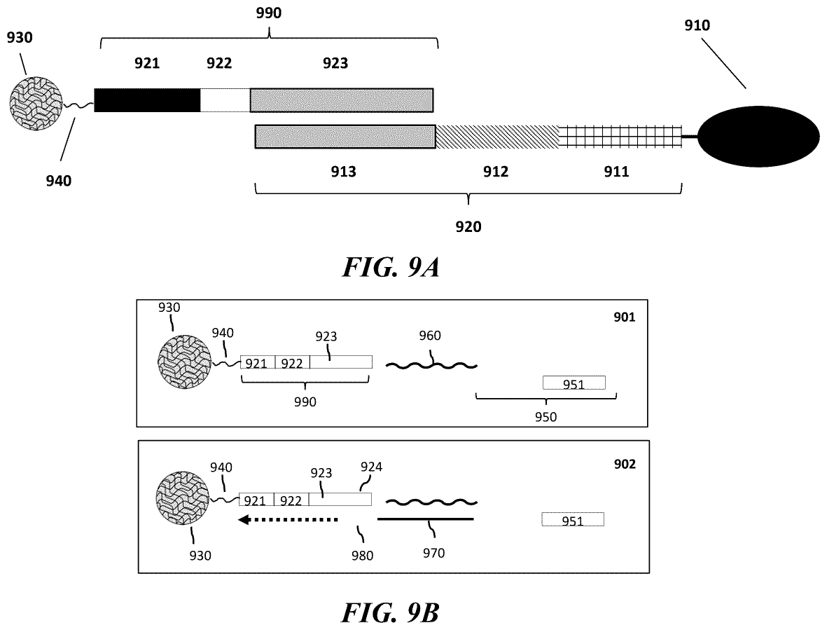

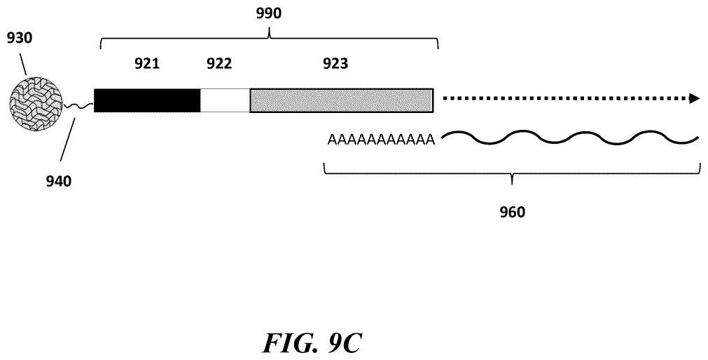

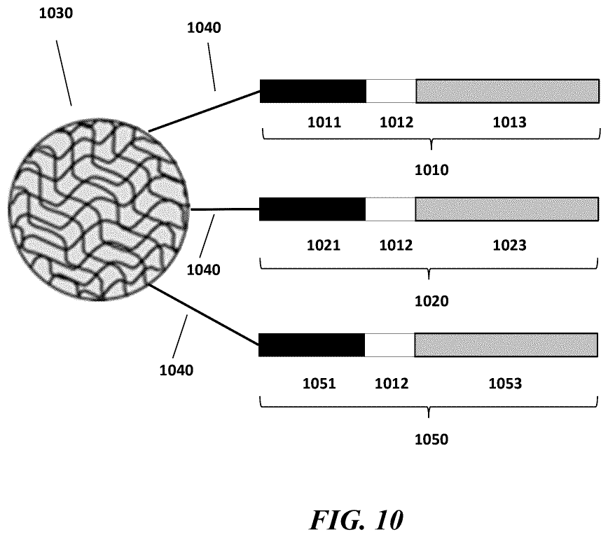

FIG. 10 illustrates another example of a barcode carrying bead. In some embodiments, analysis of multiple analytes (e.g., RNA and one or more analytes using labelling agents described herein) may comprise nucleic acid barcode molecules as generally depicted in FIG. 10. In some embodiments, nucleic acid barcode molecules 1010 and 1020 are attached to support 1030 via a releasable linkage 1040 (e.g., comprising a labile bond) as described elsewhere herein. Nucleic acid barcode molecule 1010 may comprise adapter sequence 1011, barcode sequence 1012 and capture sequence 1013, and optionally a self-complementary region (not shown). Nucleic acid barcode molecule 1020 may comprise adapter sequence 1021, barcode sequence 1012, and capture sequence 1023, and optionally a self-complementary region (not shown), wherein capture sequence 1023 comprises a different sequence than capture sequence 1013. In some instances, adapter 1011 and adapter 1021 comprise the same sequence. In some instances, adapter 1011 and adapter 1021 comprise different sequences. Although support 1030 is shown comprising nucleic acid barcode molecules 1010 and 1020, any suitable number of barcode molecules comprising common barcode sequence 1012 are contemplated herein. For example, in some embodiments, support 1030 further comprises nucleic acid barcode molecule 1050. Nucleic acid barcode molecule 1050 may comprise adapter sequence 1051, barcode sequence 1012 and capture sequence 1053, and optionally a self-complementary region (not shown), wherein capture sequence 1053 comprises a different sequence than capture sequence 1013 and 1023. In some instances, nucleic acid barcode molecules (e.g., 1010, 1020, 1050) comprise one or more additional functional sequences, such as a UMI or other sequences described herein. The nucleic acid barcode molecules 1010, 1020 or 1050 may interact with analytes as described elsewhere herein, for example, as depicted in FIGS. 9A-C.

Referring to FIG. 9A, in an instance where cells are labelled with labeling agents, capture sequence 923 may be complementary to an adapter sequence of a reporter oligonucleotide. Cells may be contacted with one or more reporter oligonucleotide 920 conjugated labelling agents 910 (e.g., polypeptide, antibody, or others described elsewhere herein). In some cases, the cells may be further processed prior to barcoding. For example, such processing steps may include one or more washing and/or cell sorting steps. In some instances, a cell that is bound to labelling agent 910 which is conjugated to oligonucleotide 920 and support 930 (e.g., a bead, such as a gel bead) comprising nucleic acid barcode molecule 990 is partitioned into a partition amongst a plurality of partitions (e.g., a droplet of a droplet emulsion or a well of a microwell array). In some instances, the partition comprises at most a single cell bound to labelling agent 910. In some instances, reporter oligonucleotide 920 conjugated to labelling agent 910 (e.g., polypeptide, an antibody, pMHC molecule such as an MHC multimer, etc.) comprises a first adapter sequence 911 (e.g., a primer sequence), a barcode sequence 912 that identifies the labelling agent 910 (e.g., the polypeptide, antibody, or peptide of a pMHC molecule or complex), and an capture handle sequence 913. Capture handle sequence 913 may be configured to hybridize to a complementary sequence, such as a capture sequence 923 present on a nucleic acid barcode molecule 990. In some instances, oligonucleotide 920 comprises one or more additional functional sequences, such as those described elsewhere herein.

Barcoded nucleic may be generated (e.g., via a nucleic acid reaction, such as nucleic acid extension or ligation) from the constructs described in FIGS. 9A-C. For example, capture handle sequence 913 may then be hybridized to complementary sequence, such as capture sequence 923 to generate (e.g., via a nucleic acid reaction, such as nucleic acid extension or ligation) a barcoded nucleic acid molecule comprising cell (e.g., partition specific) barcode sequence 922 (or a reverse complement thereof) and reporter barcode sequence 912 (or a reverse complement thereof). In some embodiments, the nucleic acid barcode molecule 990 (e.g., including “a partition-specific barcode” or “single cell barcode” molecule) further includes a UMI (not shown). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIGS. 9A-C, or a combination of workflows for an individual analyte, as described elsewhere herein. For example, by using a combination of the workflows as generally depicted in FIGS. 9A-C, multiple analytes can be analyzed.

In some instances, analysis of an analyte (e.g. a nucleic acid, a polypeptide, a carbohydrate, a lipid, etc.) comprises a workflow as generally depicted in FIG. 9A. A nucleic acid barcode molecule 990 may be co-partitioned with the one or more analytes. In some instances, nucleic acid barcode molecule 990 is attached to a support 930 (e.g., a bead, such as a gel bead), such as those described elsewhere herein. For example, nucleic acid barcode molecule 990 may be attached to support 930 via a releasable linkage 940 (e.g., comprising a labile bond), such as those described elsewhere herein. Nucleic acid barcode molecule 990 may comprise a functional sequence 921 and optionally comprise other additional sequences, for example, a barcode sequence 922 (e.g., common barcode, partition-specific barcode, or other functional sequences described elsewhere herein), and/or a UMI sequence (not shown) . . . . The nucleic acid barcode molecule 990 may comprise a capture sequence 923 that may be complementary to another nucleic acid sequence, such that it may hybridize to a particular sequence, e.g., capture handle sequence 913.

For example, capture sequence 923 may comprise a poly-T sequence and may be used to hybridize to mRNA. Referring to FIG. 9C, in some embodiments, nucleic acid barcode molecule 990 comprises capture sequence 923 complementary to a sequence of RNA molecule 960 from a cell. In some instances, capture sequence 923 comprises a sequence specific for an RNA molecule. Capture sequence 923 may comprise a known or targeted sequence or a random sequence. In some instances, a nucleic acid extension reaction may be performed, thereby generating a barcoded nucleic acid product comprising capture sequence 923, the functional sequence 921 (e.g., a region of complementarity with an additional oligonucleotide used for extension), barcode sequence 922 (e.g., two barcodes separated by a priming sequence), any other functional sequence, and a sequence corresponding to the RNA molecule 960.

In another example, capture sequence 923 may be complementary to an overhang sequence or an adapter sequence that has been appended to an analyte. For example, referring to FIG. 9B, panel 901, in some embodiments, primer 950 comprises a sequence complementary to a sequence of nucleic acid molecule 960 (such as an RNA encoding for a BCR sequence) from an biological particle. In some instances, primer 950 comprises one or more sequences 951 that are not complementary to RNA molecule 960. Sequence 951 may be a functional sequence as described elsewhere herein, for example, an adapter sequence, a sequencing primer sequence, or a sequence the facilitates coupling to a flow cell of a sequencer. In some instances, primer 950 comprises a poly-T sequence. In some instances, primer 950 comprises a sequence complementary to a target sequence in an RNA molecule. In some instances, primer 950 comprises a sequence complementary to a region of an immune molecule, such as the constant region of a TCR or BCR sequence. Primer 950 is hybridized to nucleic acid molecule 960 and complementary molecule 970 is generated (see Panel 902). For example, complementary molecule 970 may be cDNA generated in a reverse transcription reaction. In some instances, an additional sequence may be appended to complementary molecule 970. For example, the reverse transcriptase enzyme may be selected such that several non-templated bases 980 (e.g., a poly-C sequence) are appended to the cDNA. In another example, a terminal transferase may also be used to append the additional sequence. Nucleic acid barcode molecule 990 comprises a sequence 924 complementary to the non-templated bases, and the reverse transcriptase performs a template switching reaction onto nucleic acid barcode molecule 990 to generate a barcoded nucleic acid molecule comprising cell (e.g., partition specific) barcode sequence 922 (or a reverse complement thereof) and a sequence of complementary molecule 970 (or a portion thereof). In some instances, sequence 923 comprises a sequence complementary to a region of an immune molecule, such as the constant region of a TCR or BCR sequence. Sequence 923 is hybridized to nucleic acid molecule 960 and a complementary molecule 970 is generated. For example, complementary molecule 970 may be generated in a reverse transcription reaction generating a barcoded nucleic acid molecule comprising cell (e.g., partition specific) barcode sequence 922 (or a reverse complement thereof) and a sequence of complementary molecule 970 (or a portion thereof). Additional methods and compositions suitable for barcoding cDNA generated from mRNA transcripts including those encoding V (D) J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969, each of which applications is herein entirely incorporated by reference for all purposes.

In some embodiments, biological particles (e.g., cells, nuclei) from a plurality of samples (e.g., a plurality of subjects) can be pooled, sequenced, and demultiplexed by identifying mutational profiles associated with individual samples and mapping sequence data from single biological particles to their source based on their mutational profile. See, e.g., Xu J. et al., Genome Biology Vol. 20, 290 (2019); Huang Y. et al., Genome Biology Vol. 20, 273 (2019); and Heaton et al., Nature Methods volume 17, pages 615-620 (2020).

Gene expression data can reflect the underlying genome and mutations and structural variants therein. As a result, the variation inherent in the captured and sequenced RNA molecules can be used to identify genotypes de novo or used to assign molecules to genotypes that were known a priori. In some embodiments, allelic variation that is present due to haplotypic states (including linkage disequilibrium of the human leucocyte antigen loci (HLA), immune receptor loci (BCR), and other highly polymorphic regions of the genome), can also be used for demultiplexing. Expressed B cell receptors can be used to infer germline alleles from unrelated individuals, which information may be used for demultiplexing.

### Spatial Workflows

### Spatial Analysis Methods

In some aspects, provided herein are circularization-based dual 3′/5′ spatial analysis methods.

In some aspects, provided herein are methods of determining location and abundance of a nucleic acid analyte in a biological sample using a circularization-based 3′/5′ sequencing method.

In some embodiments, the circularization-based 3′/5′ spatial analysis workflow involves generating a linear extension product with a 3′ portion of the nucleic acid analyte proximal to an oligonucleotide sequence including a spatial barcode and a 5′ portion of the nucleic acid analyte distal to the oligonucleotide sequence including the spatial barcode, followed by circularization of the extension product. In some embodiments, the method includes contacting the biological sample with a substrate and hybridizing the nucleic acid analyte to a capture domain of a capture probe on an array, thereby generating a captured nucleic acid analyte. In some embodiments, the capture probe further comprises a spatial barcode.

In some embodiments, the method includes extending the capture probe using the captured nucleic acid analyte as a template to produce an extended capture probe. In some embodiments, the method further includes synthesizing a second strand using the extended capture probe as a template. Second strand synthesis can include contacting the sample with a polymerase and dNTPs. In some embodiments, the method further includes circularizing the second strand to generate a circularized second strand. In some embodiments, the circularizing the second strand is performed by ligating a 5′ terminus of the second strand to a 3′ terminus of the second strand. In some embodiments, the second strand includes a first self-complementary region at or within six nucleotides of 5′ end, and a second self-complementary region at or within six nucleotides of the 3′ end, and the method includes, prior to ligating, annealing the self-complementary regions.

In some embodiments, the method further includes determining (i) all or a part of a sequence of the captured nucleic acid analyte at its 5′ end, or a complement thereof, (ii) all or a part of a sequence of the captured nucleic acid analyte at its 3′ end, or a complement thereof, and (iii) the spatial barcode, or a complement thereof, and using the determined sequences of (i), (ii), and (iii) to determine the location and abundance of the nucleic acid analyte in the biological sample.

In some embodiments, the circularization-based 3′/5′ spatial analysis workflow involves generating a linear extension product with a 5′ end of the nucleic acid analyte proximal to an oligonucleotide sequence including a spatial barcode and a 3′ end of the nucleic acid analyte distal to the oligonucleotide sequence including the spatial barcode, followed by circularization of the extension product.

In some embodiments, all or a part of a sequence of the captured nucleic acid analyte at its 5′ end comprises about 50, about 75, about 100, about 150, about 200, about 250, about 300, about 350, about 400, about 450, about 500 or more nucleotides, and/or all or a part of a sequence of the captured nucleic acid analyte at its 3′ end comprises about 50, about 75, about 100, about 150, about 200, about 250, about 300, about 350, about 400, about 450, about 500 or more nucleotides.

In some embodiments, the method includes amplifying all or part of the circularized second strand. In some embodiments, the method includes generating amplicons each including 5′ end or 5′ portion of the nucleic acid analyte, the spatial barcode, and 3′ end or 3′ portion of the nucleic acid analyte. In some embodiments, the capture probe include two spatial barcodes and a primer site between the two spatial barcodes, and the amplifying includes amplifying

In some embodiments, determining (i) all or a part of a sequence of the captured nucleic acid analyte at its 5′ end, or a complement thereof, (ii) all or a part of a sequence of the captured nucleic acid analyte at its 3′ end, or a complement thereof, and (iii) the spatial barcode, or a complement thereof, comprises sequencing.

As previously described, spatial analysis methods provided herein, in some embodiments, include contacting the biological sample with a substrate and hybridizing the nucleic acid analyte to a capture domain of a capture probe on an array. In some embodiments, the array is on the substrate, and optionally the substrate includes glass, one or more polymers, a hydrogel, a wafer, a plate, or combinations thereof. In some embodiments, the array is on a second substrate, and optionally includes glass, one or more polymers, a hydrogel, a wafer, a plate, or combinations thereof.

In some embodiments, the method includes aligning the substrate with the second substrate comprising the array, such that at least a portion of the biological sample is aligned with at least a portion of the array. In some embodiments, the aligning includes mounting the first substrate on a first member of a support device, the first member configured to retain the first substrate. In some embodiments, the aligning further includes mounting the second substrate on a second member of the support device. In some embodiments, the aligning further includes applying a reagent medium to the first substrate and/or the second substrate. In some embodiments, the aligning includes operating an alignment mechanism of the support device to move the first member and/or the second member such that at least a portion of the biological sample is aligned with at least a portion of the array, and such that the portion of the biological sample and the portion of the array contact the reagent medium. In some embodiments, the alignment mechanism is coupled to the first member, the second member, or both the first member and the second member. In some embodiments, the alignment mechanism includes a linear actuator. The linear actuator may be configured to: move the second member along an axis orthogonal to the first member and/or the second member, move the first member along an axis orthogonal to a plane of the first member, and/or move the first member, the second member, or both the first member and the second member at a velocity of at least 0.1 mm/see, and/or move the first member, the second member, or both the first member and the second member with an amount of force of at least 0.1 lbs.

In some embodiments, at least one of the first substrate and the second substrate further includes a spacer disposed on the first substrate or the second substrate, wherein when at least the portion of the biological sample is aligned with at least a portion of the array such that the portion of the biological sample and the portion of the array contact the reagent medium, the spacer is disposed between the first substrate and the second substrate and is configured to maintain the reagent medium within a chamber formed by the first substrate, the second substrate, and the spacer, and to maintain a separation distance between the first substrate and the second substrate, wherein the spacer is positioned to surround an area on the first substrate on which the biological sample is disposed and/or the array disposed on the second substrate, wherein the area of the first substrate, the spacer, and the second substrate at least partially encloses a volume comprising the biological sample.

In some aspects, the method includes an extension reaction. Examples of extension reaction include DNA polymerization and reverse transcription. In some embodiments, the extending the capture probe utilizes a reverse transcriptase or a polymerase. Synthesizing the second strand may utilize a polymerase, such as a DNA polymerase.

In some embodiments, the method includes separating the second strand from the extended capture probe, e.g., by denaturing. In some embodiments, separating the second strand from the extended capture probe comprises adding potassium hydroxide to the substrate.

In some embodiments, circularizing the second strand utilizes a ligase, such as a PBCV-1 DNA ligase, a Chlorella virus DNA ligase, a single stranded DNA ligase, or a T4 DNA ligase.

In some embodiments, the circularized second strand comprises one or more spatial barcodes, wherein the one or more spatial barcodes comprises the spatial barcode.

In some embodiments, the circularized second strand comprises one or more functional domains, one or more unique molecule identifiers (UMIs), or combinations thereof. In some embodiments, the one or more functional domains comprise one or more primer sites. In some embodiments, the one or more primer sites amplify the captured nucleic acid analyte both at its 5′ end and at its 3′ end, thereby generating a plurality of nucleic acid analyte amplicons.

In some embodiments, plurality of nucleic acid analyte amplicons comprises: a 5′ nucleic acid analyte amplicon comprising sequences of (i) all or part of the sequence of the captured nucleic acid analyte at its 5′ end, or a complement thereof, (ii) one or more UMIs, or complements thereof, and (iii) one or more spatial barcodes, or complements thereof; and a 3′ nucleic acid analyte amplicon comprising sequences of (i) all or part of the sequence of the captured nucleic acid analyte at its 3′ end, or a complement thereof, (ii) one or more UMIs, or complements thereof. In some embodiments, the plurality of nucleic acid analyte amplicons comprises: a 5′ nucleic acid analyte amplicon comprising sequences of (i) all or part of the sequence of the captured nucleic acid analyte at its 5′ end, or a complement thereof, (ii) one UMI, or a complement thereof, and (iii) the spatial barcodes, or a complement thereof; and a 3′ nucleic acid analyte amplicon comprising sequences of (i) all or part of the sequence of the captured nucleic acid analyte at its 3′ end, or a complement thereof, (ii) one UMI, or a complement thereof, and (iii) the spatial barcodes, or a complement thereof.

In some embodiments, the capture probe further comprises one or more functional domains, a UMI, a cleavage domain, or combinations thereof. In some embodiments, the one or more functional domains comprises a primer binding site. In some embodiments, the capture domain comprises a homopolymeric sequence. In some embodiments, the capture domain comprises a poly(T) sequence.

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022) and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates. In some embodiments, the biological sample is a tissue sample, such as a tissue section. In some embodiments, the biological sample is a fresh tissue sample and/or a frozen tissue sample. In some embodiments, the biological sample is a fixed tissue sample, such as a formalin fixed paraffin embedded (FFPE) tissue sample. In some embodiments, the FFPE tissue sample is deparaffinized and decrosslinked.

In some embodiments, the biological sample is a suspension of cells or a culture of cells.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaeon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example, methanol. In some embodiments, instead of methanol, acetone or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol), is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium for antigen retrieval in the biological sample. Thus, any suitable decrosslinking agent can be used in addition, or alternatively, to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology and biomolecules. PAXgene provides a two-reagent fixative system in which tissue is firstly fixed in a solution containing methanol and acetic acid, then stabilized in a solution containing ethanol. See, Ergin B. et al., J Proteome Res. 2010 Oct. 1; 9(10):5188-96; Kap M. et al., PLOS One.; 6(11):e27704 (2011); and Mathieson W. et al., Am J Clin Pathol.; 146 (1): 25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample, is fixed, for example in methanol, acetone, acetone-methanol, PFA, PAXgene, or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes, as disclosed herein, to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663, which is herein incorporated by reference. Briefly, any of the methods described herein includes permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II) (b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II) (d) (ii) of PCT Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the substrate comprising the array of capture probes is a second substrate. In this configuration, one or more analytes or analyte derivatives (e.g., intermediate agents; e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Pub. No. 2021/0189475 and PCT Pub. Nos. WO 2021/252747 A1, WO 2022/061152 A2, and WO 2022/140028 A1, each of which is herein incorporated by reference.

FIG. 11A shows an exemplary sandwiching process 1100 where a first substrate (e.g., slide 1103), including a biological sample 1102, and a second substrate (e.g., array slide 1104 including an array having spatially barcoded capture probes 1106) are brought into proximity with one another. As shown in FIG. 11A, a liquid reagent drop (e.g., permeabilization solution 1105) is introduced on the second substrate in proximity to the capture probes 1106 and in between the biological sample 1102 and the second substrate (e.g., slide 1104 including an array having spatially barcoded capture probes 1106). The permeabilization solution 1105 may release analytes or analyte derivatives (e.g., intermediate agents; e.g., ligation products) that can be captured by the capture probes of the array 1106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 1104) is in an inferior position to the first substrate (e.g., slide 1103). In some embodiments, the first substrate (e.g., slide 1103) may be positioned superior to the second substrate (e.g., slide 1104). A reagent medium 1105 within a gap between the first substrate (e.g., slide 1103) and the second substrate (e.g., slide 1104) creates a liquid interface between the two substrates. The reagent medium may be a permeabilization solution which permeabilizes and/or digests the biological sample 1102. In some embodiments wherein the biological sample 1102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 1102 may release from the biological sample, and actively or passively migrate (e.g., diffuse) across the gap toward the capture probes on the array 1106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788 and U.S. Patent Application Pub. No. 2021/0189475, each of which is hereby incorporated by reference in its entirety.

As further shown, one or more spacers 1110 may be positioned between the first substrate (e.g., slide 1103) and the second substrate (e.g., array slide 1104 including spatially barcoded capture probes 1106). The one or more spacers 1110 may be configured to maintain a separation distance between the first substrate and the second substrate. While the one or more spacers 1110 is shown as disposed on the second substrate, the spacer may additionally or alternatively be disposed on the first substrate.

In some embodiments, the one or more spacers 1110 is configured to maintain a separation distance between first and second substrates that is between about 2 microns (μm) and about 1 mm (e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 11B shows a fully formed sandwich configuration 1125 creating a chamber 1150 formed from the one or more spacers 1110, the first substrate (e.g., the slide 1103), and the second substrate (e.g., the slide 1104 including an array 1106 having spatially barcoded capture probes) in accordance with some example implementations. In the example of FIG. 11B, the liquid reagent (e.g., the permeabilization solution 1105) fills the volume of the chamber 1150 and may create a permeabilization buffer that allows analytes (e.g., mRNA transcripts and/or other molecules) or analyte derivatives (e.g., intermediate agents; e.g., ligation products) to diffuse from the biological sample 1102 toward the capture probes of the second substrate (e.g., slide 1104). In some aspects, flow of the permeabilization buffer may deflect transcripts and/or molecules from the biological sample 1102 and may affect diffusive transfer of analytes or analyte derivatives (e.g., intermediate agents; e.g., ligation products) for spatial analysis. A partially or fully sealed chamber 1150 resulting from the one or more spacers 1110, the first substrate (e.g., slide 1103), and the second substrate (e.g., slide 1104) may reduce or prevent undesirable movement (e.g., convective movement) of transcripts and/or molecules during the diffusive transfer from the biological sample 1102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Pub. No. 2021/0189475 and PCT Publ. No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

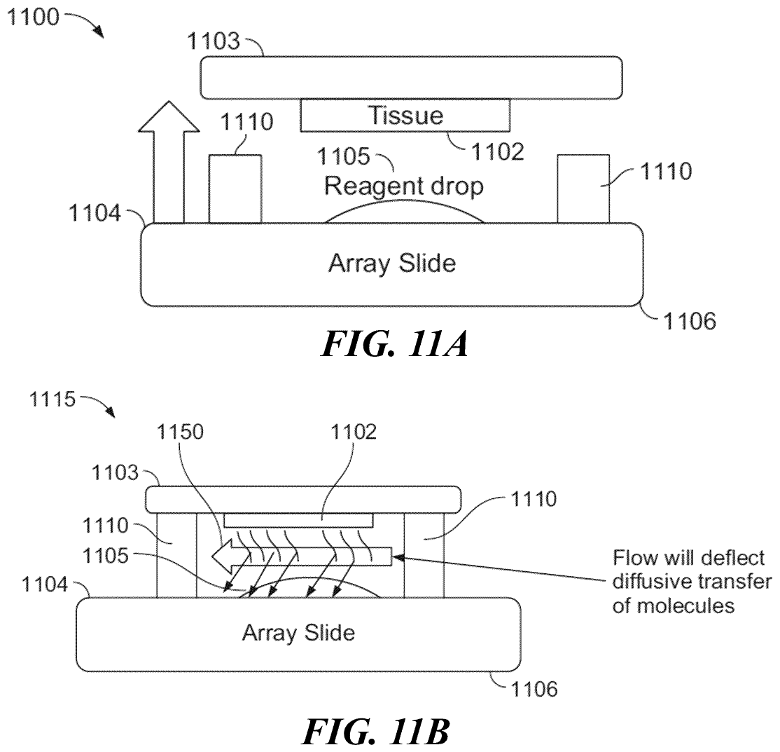

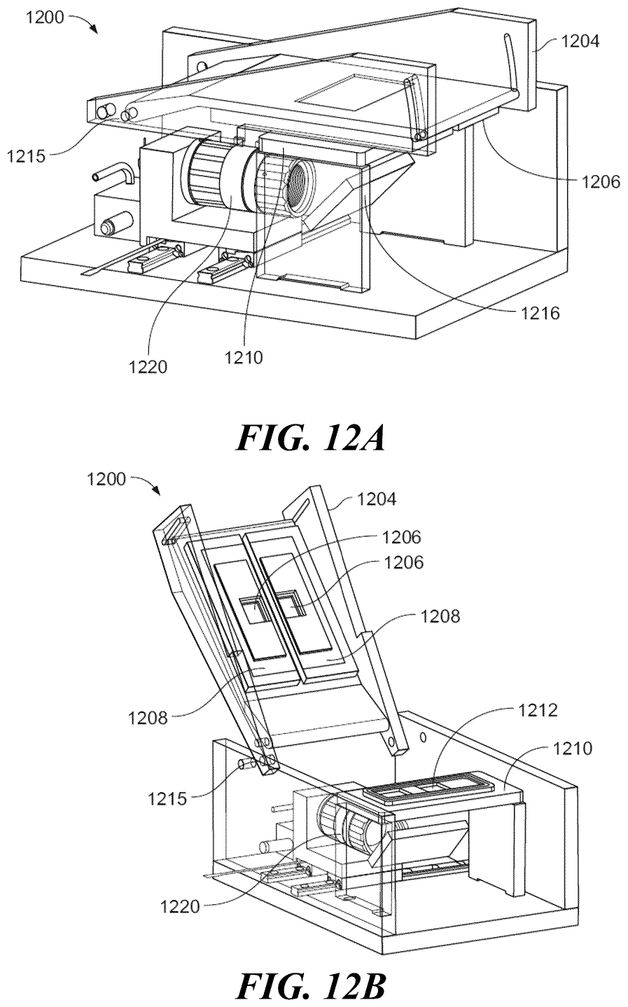

FIG. 12A is a perspective view of an example sample handling apparatus 1200 in a closed position in accordance with some example implementations. As shown, the sample handling apparatus 1200 includes a first member 1204, a second member 1210, optionally an image capture device 1220, a first substrate 1206, optionally a hinge 1215, and optionally a mirror 1216. The hinge 1215 may be configured to allow the first member 1204 to be positioned in an open or closed configuration by opening and/or closing the first member 1204 in a clamshell manner along the hinge 1215.

FIG. 12B is a perspective view of the example sample handling apparatus 1200 in an open position in accordance with some example implementations. As shown, the sample handling apparatus 1200 includes one or more first retaining mechanisms 1208 configured to retain one or more first substrates 1206. In the example of FIG. 12B, the first member 1204 is configured to retain two first substrates 1206, however the first member 1204 may be configured to retain more or fewer first substrates 1206.

In some aspects, when the sample handling apparatus 1200 is in an open position (e.g., in FIG. 12B), the first substrate 1206 and/or the second substrate 1212 may be loaded and positioned within the sample handling apparatus 1200 such as within the first member 204 and the second member 1210, respectively. As noted, the hinge 1215 may allow the first member 1204 to close over the second member 1210 and form a sandwich configuration.

In some aspects, after the first member 1204 closes over the second member 1210, an adjustment mechanism of the sample handling apparatus 1200 may actuate the first member 1204 and/or the second member 1210 to form the sandwich configuration for the permeabilization step (e.g., bringing the first substrate 1206 and the second substrate 1212 closer to each other and within a threshold distance for the sandwich configuration). The adjustment mechanism may be configured to control a speed, an angle, a force, or the like of the sandwich configuration.

In some embodiments, the biological sample (e.g., sample 1202 from FIG. 12A) may be aligned within the first member 1204 (e.g., via the first retaining mechanism 1208) prior to closing the first member 1204 such that a desired region of interest of the sample is aligned with the barcoded array of the second substrate (e.g., the slide 104 from FIG. 12A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 1206 and/or the second substrate 1212 to maintain a minimum spacing between the first substrate 1206 and the second substrate 1212 during sandwiching. In some aspects, the permeabilization solution may be applied to the first substrate 1206 and/or the second substrate 1212. The first member 1204 may then close over the second member 1210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 1220 may capture images of the overlap area between the biological sample and the capture probes on the array 106. If more than one first substrates 1206 and/or second substrates 1212 are present within the sample handling apparatus 1200, the image capture device 1220 may be configured to capture one or more images of one or more overlap areas.

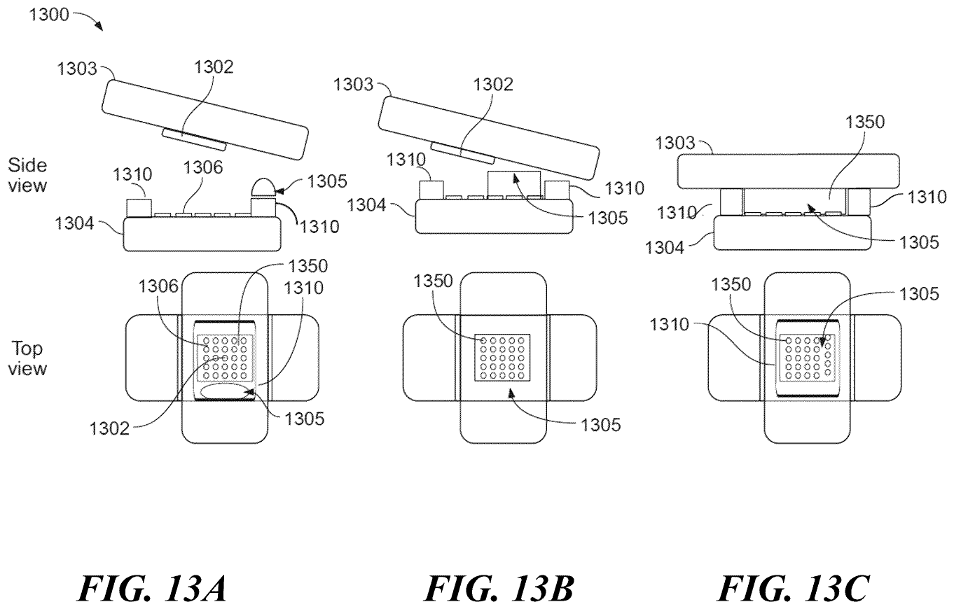

Provided herein are methods for delivering a fluid to a biological sample disposed on an area of a first substrate and an array disposed on a second substrate. FIGS. 13A-13C depict a side view and a top view of an exemplary angled closure workflow 1300 for sandwiching a first substrate (e.g., slide 1303) having a biological sample 1302 and a second substrate (e.g., slide 1304 having capture probes 1306) in accordance with some exemplary implementations.

FIG. 13A depicts the first substrate (e.g., slide 1303 including a biological sample 1302) angled over (superior to) the second substrate (e.g., slide 1304). As shown, reagent medium (e.g., permeabilization solution) 1305 is located on the spacer 1310 toward the right-hand side of the side view in FIG. 13A. While FIG. 13A depicts the reagent medium on the right-hand side of side view, it should be understood that such depiction is not meant to be limiting as to the location of the reagent medium on the spacer.

FIG. 13B shows that as the first substrate lowers and/or as the second substrate rises, the dropped side of the first substrate (e.g., a side of the slide 1303 angled toward the slide 1304) may contact the reagent medium 1305. The dropped side of the slide 303 may urge the reagent medium 1305 toward the opposite direction (e.g., towards an opposite side of the spacer 1310, towards an opposite side of the slide 1303 relative to the dropped side). For example, in the side view of FIG. 13B the reagent medium 1305 may be urged from right to left as the sandwich is formed.

In some embodiments, the first substrate and/or the second substrate are further moved to achieve an approximately parallel arrangement of the first substrate and the second substrate.

FIG. 13C depicts a full closure of the sandwich between the first substrate and the second substrate with the spacer 1310 contacting both the first substrate and the second substrate and maintaining a separation distance and optionally the approximately parallel arrangement between the two substrates. As shown in the top view of FIG. 13C, the spacer 1310 fully encloses and surrounds the biological sample 1302 and the capture probes 1306, and the spacer 1310 form the sides of chamber 1350 which holds a volume of the reagent medium 1305.

While FIG. 13C depicts the first substrate (e.g., the slide 1303 including biological sample 1302) angled over (superior to) the second substrate (e.g., slide 1304) and the second substrate comprising the spacer 1310, it should be understood that an exemplary angled closure workflow can include the second substrate angled over (superior to) the first substrate and the first substrate comprising the spacer 1310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 1303 and slide 1304) during a permeabilization step. In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 1303 and the slide 1304), an angled closure workflow may be used to suppress or eliminate bubble formation.

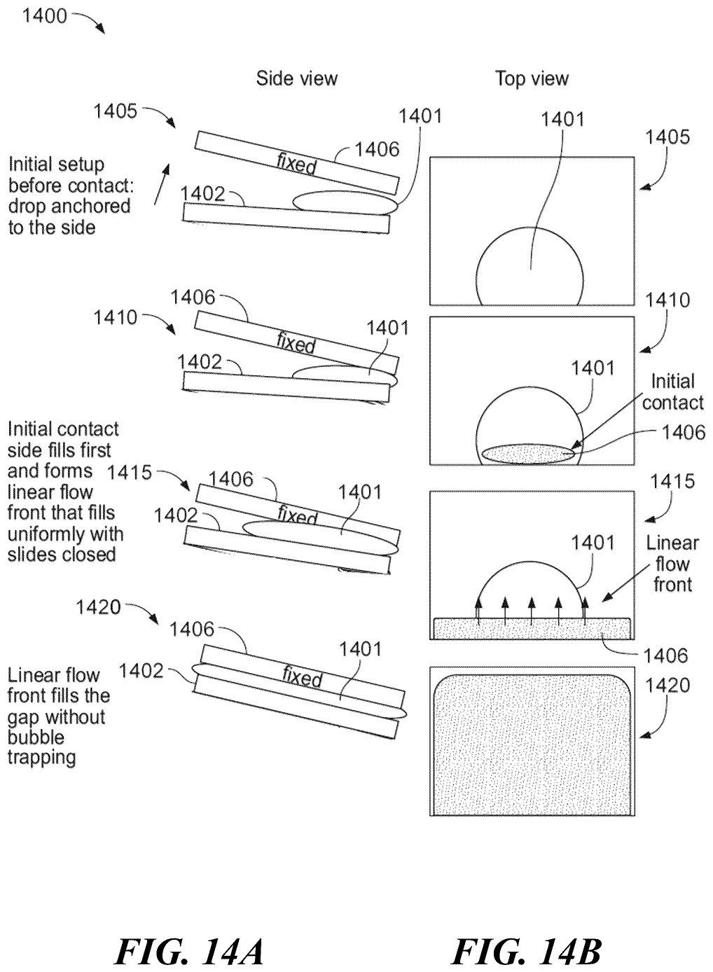

FIG. 14A is a side view of the angled closure workflow 1400 in accordance with some exemplary implementations. FIG. 14B is a top view of the angled closure workflow 1400 in accordance with some exemplary implementations. As shown at step 1405, reagent medium 1401 is positioned to the side of the substrate 1402.

At step 1410, the dropped side of the angled substrate 406 contacts the reagent medium 401 first. The contact of the substrate 1406 with the reagent medium 401 may form a linear or low curvature flow front that fills the gap between the two substrates 406 and 402 uniformly with the slides closed.

At step 1414, the substrate 1406 is further lowered toward the substrate 1402 (or the substrate 1402 is raised up toward the substrate 1406) and the dropped side of the substrate 406 may contact and urge the reagent medium toward the side opposite the dropped side, thereby creating a linear or low curvature flow front that may prevent or reduce bubble trapping between the substrates.

At step 1420, the reagent medium 1401 fills the gap between the substrate 1406 and the substrate 1402. The linear flow front of the liquid reagent may be formed by squeezing the reagent medium 1401 volume along the contact side of the substrate 1402 and/or the substrate 1406. Additionally, capillary flow may also contribute to filling the gap area.

In some embodiments, the reagent medium comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, SDS), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and SDS. More generally, chemical lysis agents can include, without limitation, organic solvents, chelating agents, detergents, surfactants, and chaotropic agents. In some embodiments, the reagent medium comprises a protease.

Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of SDS or a sodium salt thereof, proteinase K, pepsin, N-lauroylsarcosine, and RNase.

In some embodiments, the reagent medium comprises polyethylene glycol (PEG). In some embodiments, the PEG molecular weight is from about 2K to about 16K. In some embodiments, the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II) (b) (vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe, thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II) (a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II) (g) of PCT Publication No. WO2020/176788 and/or U.S. Patent Application Publication No. 2020/0277663, which is herein incorporated by reference. Some quality control measures are described in Section (II) (h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II) (c) of PCT Publication No. WO2020/176788 and/or U.S. Patent Application Publication No. 2020/0277663, which is herein incorporated by reference. Exemplary features and geometric attributes of an array can be found in Sections (II) (d) (i), (II) (d) (iii), and (II) (d) (iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II) (e) of PCT Publication No. WO2020/176788 and/or U.S. Patent Application Publication No. 2020/0277663, which is herein incorporated by reference.

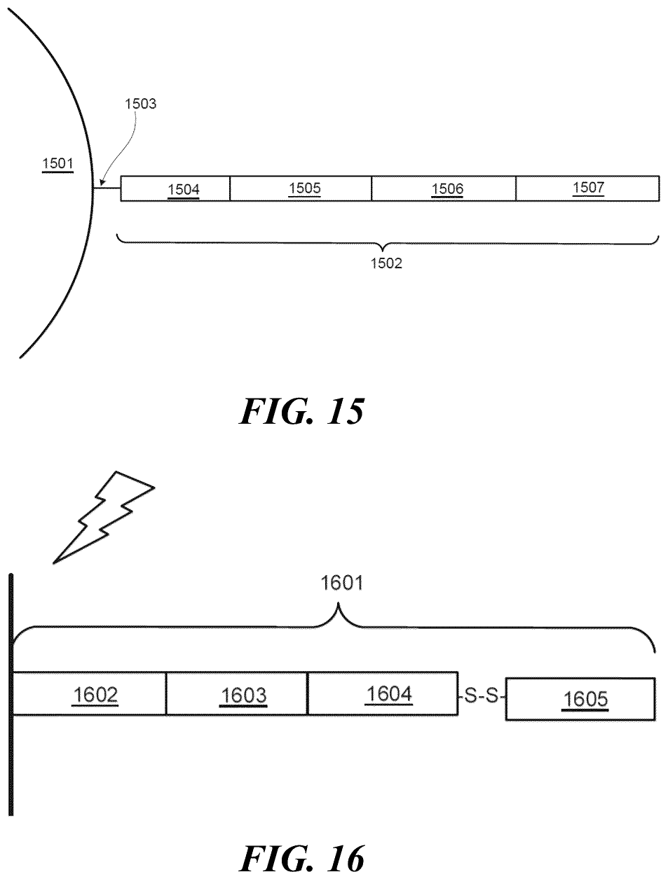

FIG. 15 is a schematic diagram showing an exemplary capture probe, as described herein. As shown, the capture probe 1502 is optionally coupled to a feature 1501 by a cleavage domain 1503, such as a disulfide linker. The capture probe can include a functional sequence 1504 that is useful for subsequent processing. The functional sequence 1504 can include all or a part of sequencer specific flow cell attachment sequence (e.g., a P5 or P7 sequence), all or a part of a sequencing primer sequence, (e.g., a R1 primer binding site, a R2 primer binding site), a region of complementarity with an additional oligonucleotide used in an extension reaction, or combinations thereof. The capture probe can also include a spatial barcode 1505. The capture probe can also include a unique molecular identifier (UMI) sequence 1506. While FIG. 15 shows the spatial barcode 1505 as being located upstream (5′) of UMI sequence 1506, it is to be understood that capture probes wherein UMI sequence 1506 is located upstream (5′) of the spatial barcode 1505 is also suitable for use in any of the methods described herein. The capture probe can also include a capture domain 1507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

FIG. 16 is a schematic illustrating a cleavable capture probe, wherein the cleaved capture probe can enter into a non-permeabilized cell and bind to analytes within the cell. The capture probe 601 can contain a cleavage domain 1602, a cell penetrating peptide 1603, a reporter molecule 1604, and a disulfide bond (—S—S—). 1605 represents all other parts of a capture probe, for example, a spatial barcode, a self-complementary region, and a capture domain.

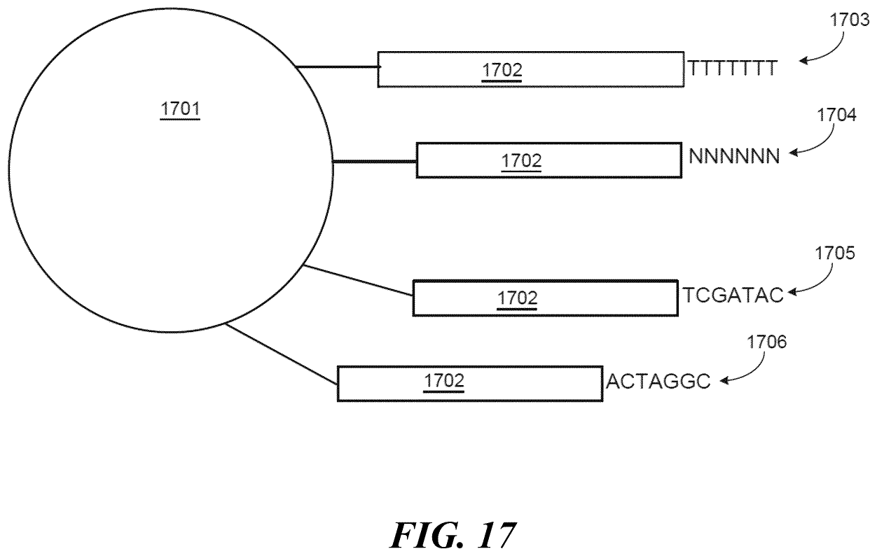

FIG. 17 is a schematic diagram of an exemplary multiplexed spatially-barcoded feature. In FIG. 17, the feature 1701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 1702. One type of capture probe associated with the feature can include the spatial barcode 1702 in combination with a poly(T) capture domain 1703, designed to capture mRNA target analytes. A second type of capture probe associated with the feature can include the spatial barcode 1702 in combination with a random N-mer capture domain 1704 for gDNA analysis. A third type of capture probe associated with the feature can include the spatial barcode 1702 in combination with a capture domain complementary to the analyte capture agent of interest 1705. A fourth type of capture probe associated with the feature can include the spatial barcode 1702 in combination with a capture probe that can specifically bind a nucleic acid molecule 1706 that can function in a CRISPR assay (e.g., CRISPR/Cas9). While only four different capture probe-barcoded constructs are shown in FIG. 17, capture-probe barcoded constructs can be tailored for analyses of any given analyte associated with a nucleic acid and capable of binding with such a construct. For example, the schemes shown in FIG. 17 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 (e.g., self-complementary region) are common to all of the probes attached to a given feature. In some embodiments, the UMI sequence 506 of a capture probe attached to a given feature is different from the UMI sequence of a different capture probe attached to the given feature.

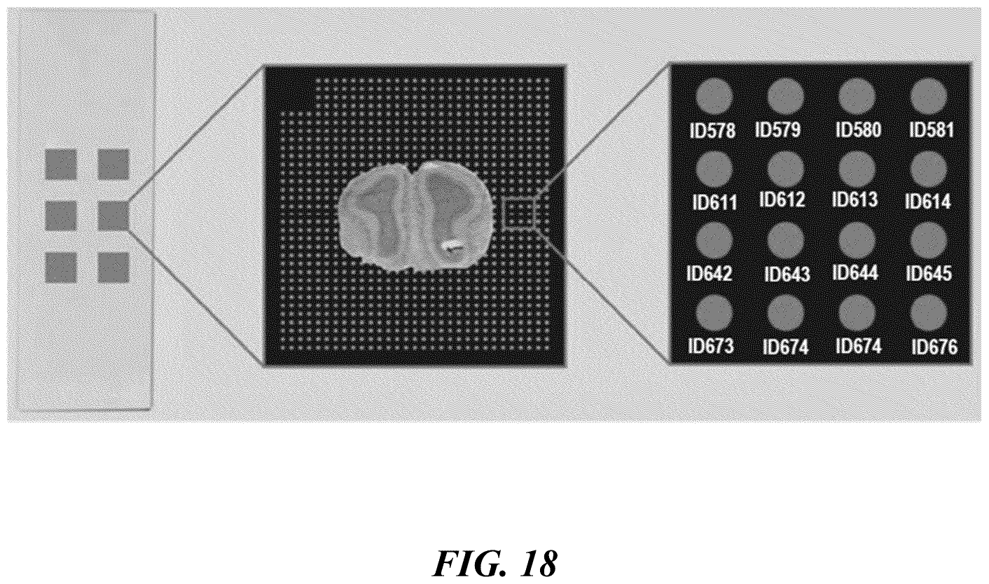

FIG. 18 depicts an exemplary arrangement of barcoded features within an array. From left to right, FIG. 18 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128, which is herein incorporated by reference in its entirety. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture probe binding domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA), which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the captured analytes are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022) and/or the Visium Spatial Gene Expression Reagent Kits-Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II) (e) (ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a sealable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### EXAMPLES

The following examples are included for illustrative purposes only and are not intended to limit the scope of the present disclosure.

### Example 1: Single Cell Circularization-Based Dual 3′/5′ Assay

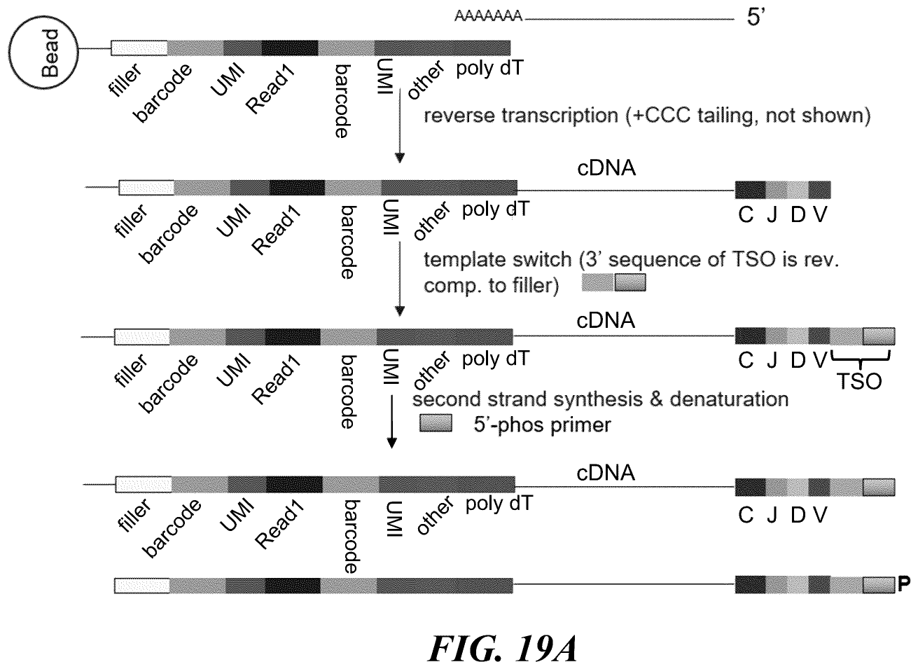

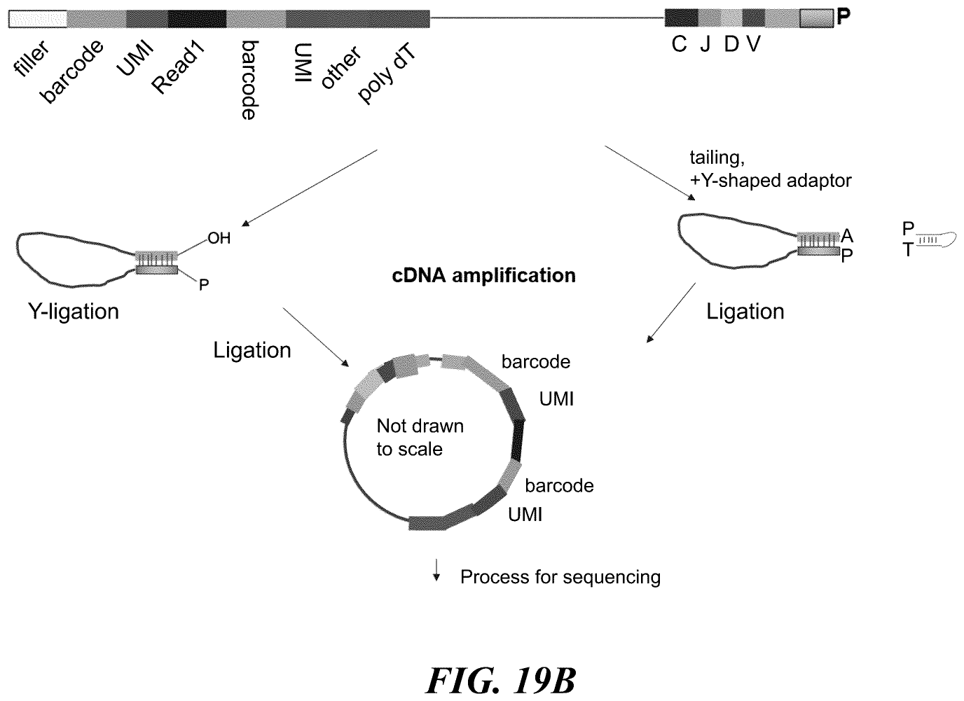

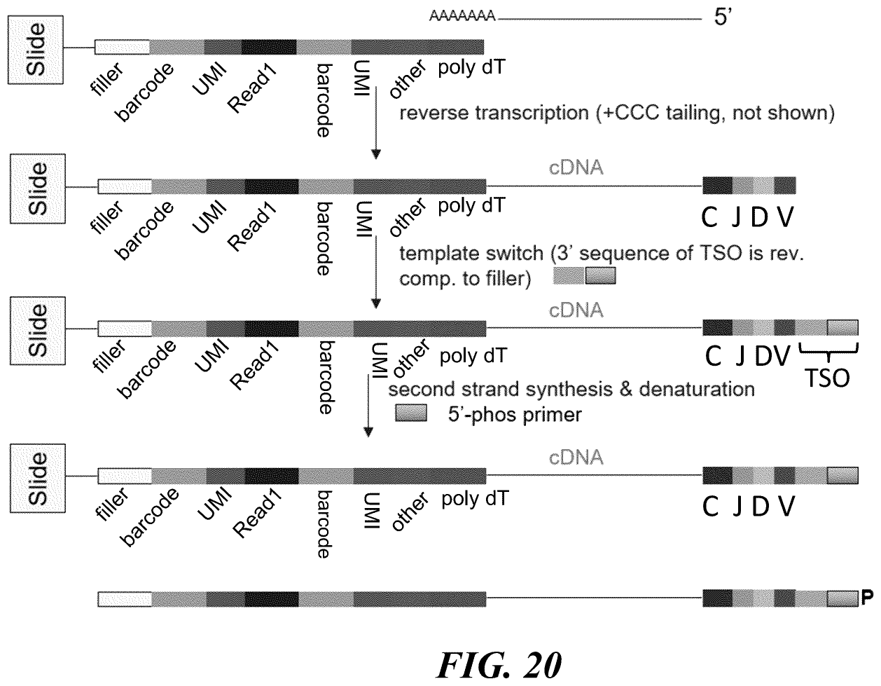

This example provides a single cell circularization based dual 3′/5′ sequencing assay. FIG. 19A shows an example of generating an extended product for circularization from an immune receptor mRNA nucleic acid analyte. In the partition (e.g., droplet or well), a barcode nucleic acid molecule attached to a gel bead including a 1^{st} self-complementary region (shown as “filler”), two partition-specific barcodes and two UMIs separating a primer site (“read 1”), and polydT) is used as a primer for reverse transcription of the mRNA, following hybridizing with 3′ polyA of the mRNA. The reverse transcriptase adds a non-templated polyC to the end of the resulting cDNA. In this example, the full mRNA is reverse transcribed and a 5′ portion of the sequence includes a VDJ region. Next, a template-switching oligonucleotide including a polyG and a 2^{nd} self-complementary region (the reverse complement of the region “filler” as shown) is used to complete generation of the extended product. The extension product includes in order, a self-complementary region, two barcodes and UMIs separated by a primer region, a sequence of the mRNA in 3′ to 5′ direction, and an additional self-complementary region. Finally, a phosphorylated primer is used to generate a second strand of the extension product, and denaturation is performed to remove the second strand extension product from the first strand extension product.

Next, circularization of the second strand extension product is performed. As shown in FIG. 19B, 1^{st} self-complementary and 2^{nd} self-complementary regions are annealed to bring together the ligatable ends. FIG. 19B, middle left shows a Y-ligation to ligate a 5′ terminal phosphate of the second strand extension product to a 3′ terminal hydroxy of the second strand extension product. FIG. 19B, middle right shows ligation of a short (fewer than 10 base pairs) self-annealed oligo having a free 5′ phosphate and free 3′ hydroxy for ligation to 3′ hydroxy and 5′ phosphate of the second strand extension product. Either ligation method results in a circularized extension product (FIG. 19B, bottom panel). Next, the circularized extension product is processed for sequencing. For example, cDNA amplification may be performed, by using a forward primer and a reverse primer that both bind to the primer region (shown as “read 1”) there by generating linear amplicons of substantially the whole circularized extended product. To generate the sequencing library, the cDNA amplification product may be fragmented, repaired, and appended with sequencing primers. This method allows for obtaining sequences from fragments including a 3′ end and the barcode, and fragments including a 5′ end and the barcode.

### Example 2: Spatial Circularization-Based Dual 3′/5′ Assay

This example provides a spatial circularization-based dual 3′/5′ sequencing assay. FIG. 19A shows an example of generating an extended product for circularization from an immune receptor mRNA nucleic acid analyte. A barcode nucleic acid molecule, of a spatial array and attached to a substrate, and including a 1^{st} self-complementary region (shown as “filler”), two spatial barcodes (specific to a location on the array) and two UMIs separating a primer site (“read 1”), and polydT, is used as a primer for reverse transcription of the mRNA, following hybridizing with 3′ polyA of the mRNA. The reverse transcriptase adds a non-templated polyC to the end of the resulting cDNA. In this example, the full mRNA is reverse transcribed and a 5′ portion of the sequence includes a VDJ region. Next, a template-switching oligonucleotide including a polyG and a 2^{nd} self-complementary region (the reverse complement of the region “filler” as shown) is used to complete generation of the extended product. Finally, a phosphorylated primer is used to generate a second strand of the extension product, and denaturation is performed to remove the second strand extension product from the first strand extension product.

Next, circularization of the second strand extension product is performed. The circularization step for the spatial assay may be performed similarly to the steps described in Example 2 and shown in FIG. 19B.

### Example 3: Circularization-Based Dual 3′/5′ Assay of an Alternative Orientation

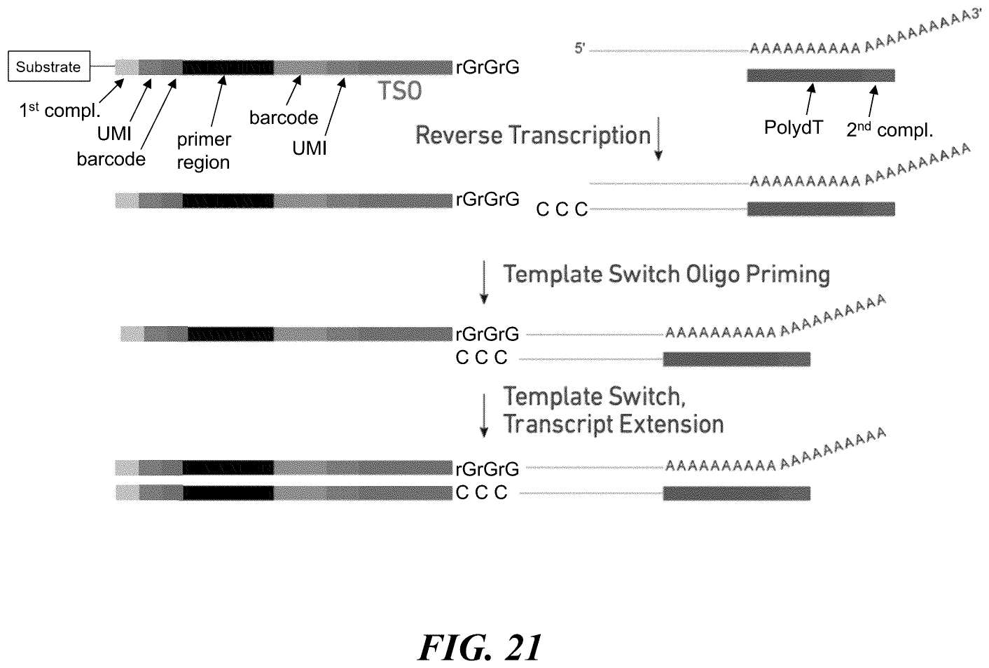

This example provides a circularization-based dual 3′/5′ sequencing assay having the nucleic acid analyte in an alternative orientation. FIG. 21 shows an example of generating an extended product for circularization from an immune receptor mRNA nucleic acid analyte. An oligonucleotide molecule attached to a substrate and including a polydT region and a self-complementary region is used as a primer for reverse transcription of the mRNA, following hybridizing with 3′ polyA of the mRNA. The reverse transcriptase adds a non-templated polyC to the end of the resulting cDNA. Next, a barcode nucleic acid molecule, attached to a gel bead and including an additional self-complementary region, two partition-specific barcodes and two UMIs separating a primer region, and a TSO region including a polydG, hybridizes to the polyC region and an extension product is generated. The extension product includes in order, a self-complementary region, two barcodes and UMIs separated by a primer region, a TSO region, a sequence of the mRNA in 5′ to 3′ direction, and an additional self-complementary region.

Next, circularization the extension product may be performed by the same method described in Example 2 and as shown in FIG. 19B.

What is claimed is:

1. A method of preparing a sequencing library comprising:
(a) providing:
i. a biological sample comprising a nucleic acid analyte,
ii. a first oligonucleotide comprising at least one barcode sequence, a region that hybridizes to a first portion of the nucleic acid analyte or an extension product thereof, and a first region for self-complementarity, and
iii. a second oligonucleotide comprising a region that hybridizes to a second portion of the nucleic acid analyte or extension product thereof, a second region for self-complementarity, and a primer binding site;
(b) performing extension reactions comprising:
i. an extension reaction using the first oligonucleotide and the nucleic acid analyte or an extension product thereof, and
ii. an extension reaction using the second oligonucleotide and the nucleic acid analyte or an extension product thereof,
wherein following the extension reactions, an extended molecule is generated comprising: a sequence of the first oligonucleotide comprising the at least one barcode sequence and the first region for self-complementarity, a sequence of the nucleic acid analyte comprising the first portion and the second portion, and a sequence of the second oligonucleotide comprising the second region for self-complementarity;
(c) annealing the first region of self-complementarity to the second region of self-complementarity;
(d) ligating a 5′ terminus and a 3′ terminus of the extended molecule to generate a circularized barcoded nucleic acid molecule; and
(e) performing an amplification reaction to generate amplicons.

2. The method of claim 1, wherein the at least one barcode sequence comprises a first barcode sequence and a second barcode sequence, and wherein the first oligonucleotide comprises a primer region positioned between the first barcode sequence and the second barcode sequence.

3. The method of claim 1, further comprising (f) fragmenting the amplicons to generate first fragments comprising a sequence of the first end of the nucleic acid analyte and the first barcode, and second fragments comprising a sequence of the second end of the nucleic acid analyte and the second barcode.

4. The method of claim 1, wherein the method further includes generating a sequencing library.

5. The method of claim 2, wherein the first barcode sequence and the second barcode sequence are identical.

6. The method of claim 1, wherein the first oligonucleotide further comprises at least one unique molecular identifier.

7. The method of claim 6, wherein the at least one unique molecular identifier comprises two unique molecular identifiers.

8. The method claim 1, wherein the extension reaction using the first oligonucleotide is performed before the extension reaction using the second oligonucleotide.

9. The method of claim 1, wherein the nucleic acid analyte is an mRNA.

10. The method of claim 9, wherein the region that hybridizes to the first portion of the mRNA or extension product thereof comprises a polyT sequence and the first portion of the mRNA comprises a 3′ polyA sequence.

11. The method of claim 9, wherein the region that hybridizes to the second portion of the mRNA or extension product thereof comprises a polyG sequence, and wherein the mRNA or extension product thereof is the extension product and comprises a non-templated terminal polyC.

12. The method of claim 10, wherein the extensions reaction using the second oligonucleotide is performed before the extension reaction using the first oligonucleotide.

13. The method of claim 9, wherein the region that hybridizes to the second portion of the mRNA or extension product thereof comprises a polyT sequence and the second portion of the mRNA comprises a 3′ polyA sequence.

14. The method of claim 9, wherein the region that hybridizes to the first portion of the mRNA or extension product thereof comprises a polyG sequence, and wherein the mRNA or extension product thereof is the extension product and the first portion of the extension product comprises a non-templated terminal polyC.

15. The method of claim 1, wherein the first oligonucleotide is part of an array.

16. The method of claim 1, wherein the first oligonucleotide is attached to a substrate.

17. The method of claim 16, wherein the substrate comprises glass, one or more polymers, a hydrogel, a wafer, a plate, or combinations thereof.

18. The method claim 15, wherein the biological sample is a cell or tissue sample attached to a support, and the at least one barcode is a spatial barcode.

19. The method of claim 16, wherein the substrate comprises a bead, a surface of a well, or a slide.

20. The method claim 1, wherein the biological sample is a single cell, cell bead, or nuclei, and the biological sample is provided in a partition.

21. The method of claim 20, wherein the at least one barcode is a partition-specific barcode.

22. The method of claim 1, wherein after (c) and prior to (d), the method further comprises contacting the self-complementary barcoded cDNA molecule with a phosphorylated primer and extending from the phosphorylated primer to generate an extension product of the self-complementary barcoded cDNA.

23. The method of claim 1, wherein the sequence of the nucleic acid analyte is at least 100 nucleotides in length.

24. The method of claim 23, wherein following the extension reactions, the extended molecule generated comprises in 3′ to 5′ or 5′ to 3′ order: the sequence of the first oligonucleotide, the nucleic acid analyte sequence in 3′ to 5′ orientation with respect to the nucleic acid analyte, and the sequence of the second oligonucleotide, wherein a 5′ end of the nucleic acid analyte sequence is adjacent to the sequence of the second oligonucleotide.

25. The method of claim 24, wherein the sequence of the nucleic acid analyte is at least 100 nucleotides in length and following the ligating, 5′ end of the nucleic acid analyte sequence is at a proximity of at least 50 nucleotides from a barcode sequence of the at least one barcode sequence in the circularized barcoded nucleic acid molecule.

26. The method of claim 23, wherein following the extension reactions, the extended molecule generated comprises in order: the sequence of the first oligonucleotide, the nucleic acid analyte sequence in 5′ to 3′ orientation with respect to the nucleic acid analyte, and the sequence of the second oligonucleotide, optionally wherein the nucleic acid analyte is an mRNA and the polyA sequence of the mRNA is adjacent to the sequence of the second oligonucleotide.

27. The method of claim 26, wherein the sequence of the mRNA is at least 100 nucleotides in length and following the ligating, the polyA sequence of the mRNA sequence is at a proximity of at least 50 nucleotides from a barcode sequence of the at least one barcode sequence in the circularized barcoded nucleic acid molecule.
